(19) United States
(12) Patent Application Publication
(10) Pub. No.: US 2018/0314842 A1
(43) Pub. Date: Nov. 1, 2018

(54) COMPUTING SYSTEM WITH GENOMIC INFORMATION ACCESS MECHANISM AND METHOD OF OPERATION THEREOF

(71) Applicant: Awakens, Inc., San Francisco, CA (US)

(72) Inventor: Kensuke Numakura, Funabashi-shi (JP)

(21) Appl. No.: 15/961,536

(22) Filed: Apr. 24, 2018

Related U.S. Application Data
(60) Provisional application No. 62/490,735, filed on Apr. 27, 2017.

(51) Int. Cl. G06F 21/60; G06F 19/28; G06F 17/30

(52) U.S. Cl. CPC G06F 21/604; G06F 19/28; C12Q 1/6869; G06F 17/30005; G06F 17/30174; G06F 17/30985

(57) ABSTRACT

A method of operation of a computing system includes registering different instances of a genomic raw data for a user profile; generating a conversion genomic data for each of the genomic raw data by removing a genomic raw line of the genomic raw data for reducing a genomic data size; generating a unification genomic file with a control unit based on a merge policy for merging different instances of a genotype sample from each of the conversion genomic data; and retrieving a personal genomic data based on the unification genomic file for presenting an interpretation data on a device.

### CROSS-REFERENCE TO RELATED APPLICATION(S)

This application claims the benefit of U.S. Provisional Patent Application Ser. No. 62/490,735 filed Apr. 27, 2017, and the subject matter thereof is incorporated herein by reference thereto.

### TECHNICAL FIELD

The present invention relates generally to a computing system, and more particularly to a system with genomic information access mechanism.

### BACKGROUND ART

Modern portable consumer and industrial electronics, especially client devices such as cellular phones, portable digital assistants, and combination devices, are providing increasing levels of functionality to support modern life including location-based information services. Research and development in the existing technologies can take a myriad of different directions.

As users become more empowered with the growth of mobile based service devices, new and old paradigms begin to take advantage of this new device space. There are many technological solutions to take advantage of this new device opportunity. One existing approach is to use location information to provide navigation services such as a global positioning system (GPS) for a car or on a mobile device such as a cell phone, portable navigation device (PND) or a personal digital assistant (PDA). Another existing approach is to collect personal information to provide financial, education, and health care services using the mobile device.

Mobile devices allow users to create, transfer, store, and/or consume information in order for users to create, transfer, store, and consume in the “real world.” One such use of mobile device services is to efficiently transfer user information to provide user specific services.

Computing systems and personalized services enabled systems have been incorporated in automobiles, notebooks, handheld devices, and other portable products. Today, these systems aid users by incorporating available, real-time relevant information, such as maps, directions, local businesses, or other points of interest (POI) to be accessed from locations where network connectivity is allowed. The real-time information provides invaluable relevant information.

However, a computing system improving a mechanism to access genomic information has become a paramount concern for the consumer. The inability decreases the benefit of using the tool.

Thus, a need still remains for a computing system with genomic information access mechanism from a personal mobile device. In view of the increasing mobility of the workforce and social interaction, it is increasingly critical that answers be found to these problems. In view of the ever-increasing commercial competitive pressures, along with growing consumer expectations and the diminishing opportunities for meaningful product differentiation in the marketplace, it is critical that answers be found for these problems. Additionally, the need to reduce costs, improve efficiencies and performance, and meet competitive pressures adds an even greater urgency to the critical necessity for finding answers to these problems. Solutions to these problems have been long sought but prior developments have not taught or suggested any solutions and, thus, solutions to these problems have long eluded those skilled in the art.

### DISCLOSURE OF THE INVENTION

The present invention provides a method of operation of a computing system including: registering different instances of a genomic raw data for a user profile; generating a conversion genomic data for each of the genomic raw data by removing a genomic raw line of the genomic raw data for reducing a genomic data size; generating a unification genomic file with a control unit based on a merge policy for merging different instances of a genotype sample from each of the conversion genomic data; and retrieving a personal genomic data based on the unification genomic file for presenting a phenotype data, an interpretation data, or a combination thereof on a device.

The present invention provides a computing system, including: a control unit for: registering different instances of a genomic raw data for a user profile; generating a conversion genomic data for each of the genomic raw data by removing a genomic raw line of the genomic raw data for reducing a genomic data size; generating a unification genomic file based on a merge policy for merging different instances of a genotype sample from each of the conversion genomic data; retrieving a personal genomic data based on the unification genomic file; and a communication unit, coupled to the control unit, for transmitting the personal genomic data for presenting a phenotype data, an interpretation data, or a combination thereof on a device.

The present invention provides a computing system having a non-transitory computer readable medium including instructions for execution, the instructions comprising: registering different instances of a genomic raw data for a user profile; generating a conversion genomic data for each of the genomic raw data by removing a genomic raw line of the genomic raw data for reducing a genomic data size; generating a unification genomic file with a control unit based on a merge policy for merging different instances of a genotype sample from each of the conversion genomic data; and retrieving a personal genomic data based on the unification genomic file for presenting a phenotype data, an interpretation data, or a combination thereof on a device.

Certain embodiments of the invention have other steps or elements in addition to or in place of those mentioned above. The steps or element will become apparent to those skilled in the art from a reading of the following detailed description when taken with reference to the accompanying drawings.

### BRIEF DESCRIPTION OF THE DRAWINGS

FIG. 1 is a computing system with genomic information access mechanism in an embodiment of the present invention.

FIG. 2 is an example of a first example of a registration process for the computing system.

FIG. 3 is a second example of a registration process for the computing system.

FIG. 4 is an example of the genomic raw data.

FIG. 5 is various examples of genomic information.

FIG. 6 is an example of system architecture of the computing system.

FIG. 7 is an example of system architecture for encrypting the genomic information.

FIG. 8 is an example of system architecture for retrieving the genomic information.

FIG. 9 is an example of retrieving an interpretation data.

FIG. 10 is an example of a display example of the personal genomic data.

FIG. 11 is an exemplary block diagram of the computing system.

FIG. 12 is a control flow of the computing system.

FIG. 13 is a flow chart of the conversion module.

FIG. 14 a flow chart of the format module.

FIG. 15 a flow chart of the reference module.

FIG. 16 a flow chart of the multi module.

FIG. 17 a first flow chart of the retriever module.

FIG. 18 a second flow chart of the retriever module.

FIG. 19 is a flow chart of a method of operation of the computing system in a further embodiment of the present invention.

### BEST MODE FOR CARRYING OUT THE INVENTION

The following embodiments are described in sufficient detail to enable those skilled in the art to make and use the invention. It is to be understood that other embodiments would be evident based on the present disclosure, and that system, process, or mechanical changes may be made without departing from the scope of the present invention.

In the following description, numerous specific details are given to provide a thorough understanding of the invention. However, it will be apparent that the invention may be practiced without these specific details. In order to avoid obscuring the present invention, some well-known circuits, system configurations, and process steps are not disclosed in detail.

The drawings showing embodiments of the computing system are semi-diagrammatic and not to scale and, particularly, some of the dimensions are for the clarity of presentation and are shown exaggerated in the drawing FIGs. Similarly, although the views in the drawings for ease of description generally show similar orientations, this depiction in the FIGs. is arbitrary for the most part. Generally, the invention can be operated in any orientation. The embodiments have been numbered first embodiment, second embodiment, etc. as a matter of descriptive convenience and are not intended to have any other significance or provide limitations for the present invention.

One skilled in the art would appreciate that the format with which navigation information is expressed is not critical to some embodiments of the invention. For example, in some embodiments, navigation information is presented in the format of (X, Y), where X and Y are two ordinates that define the geographic location, i.e., a position of a user.

In an alternative embodiment, navigation information is presented by longitude and latitude related information. In a further embodiment of the present invention, the navigation information also includes a velocity element including a speed component and a heading component.

The term “relevant information” referred to herein includes the navigation information described as well as information relating to points of interest to the user, such as local business, hours of businesses, types of businesses, advertised specials, traffic information, maps, local events, and nearby community or personal information.

The term “module” referred to herein can include software, hardware, or a combination thereof in the present invention in accordance with the context in which the term is used. For example, the software can be machine code, firmware, embedded code, and application software. Also for example, the hardware can be circuitry, processor, computer, integrated circuit, integrated circuit cores, a pressure sensor, an inertial sensor, a microelectromechanical system (MEMS), passive devices, or a combination thereof. Further, if a module is written in the apparatus claims section below, the modules are deemed to include hardware circuitry for the purposes and the scope of apparatus claims.

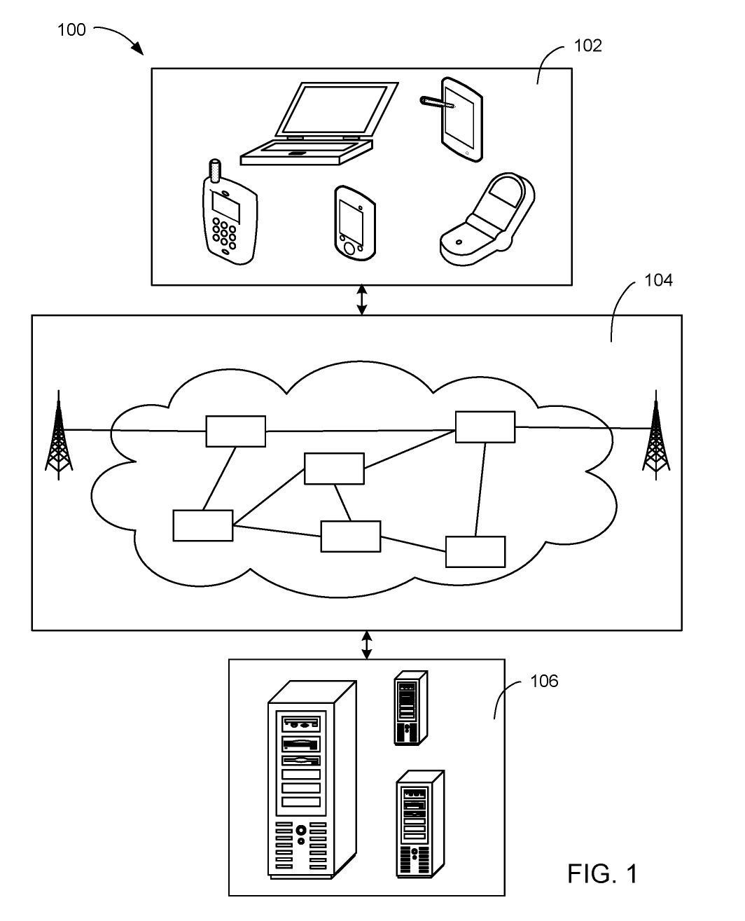

Referring now to FIG. 1, therein is shown a computing system 100 with genomic information access mechanism in an embodiment of the present invention. The computing system 100 includes a first device 102, such as a client or a server, connected to a second device 106, such as a client or server, with a communication path 104, such as a wireless or wired network.

For example, the first device 102 can be of any of a variety of mobile devices, such as a cellular phone, personal digital assistant, a notebook computer, automotive telematic computing system, a head unit, or other multi-functional mobile communication or entertainment device. The first device 102 can be a standalone device, or can be incorporated with a vehicle, for example a car, truck, bus, or train. The first device 102 can couple to the communication path 104 to communicate with the second device 106.

For illustrative purposes, the computing system 100 is described with the first device 102 as a mobile computing device, although it is understood that the first device 102 can be different types of computing devices. For example, the first device 102 can also be a non-mobile computing device, such as a server, a server farm, or a desktop computer. In another example, the first device 102 can be a particularized machine, such as a mainframe, a server, a cluster server, rack mounted server, or a blade server, or as more specific examples, an IBM System z10™ Business Class mainframe or a HP ProLiant ML™ server.

The second device 106 can be any of a variety of centralized or decentralized computing devices. For example, the second device 106 can be a computer, grid computing resources, a virtualized computer resource, cloud computing resource, routers, switches, peer-to-peer distributed computing devices, or a combination thereof.

The second device 106 can be centralized in a single computer room, distributed across different rooms, distributed across different geographical locations, embedded within a telecommunications network. The second device 106 can have a means for coupling with the communication path 104 to communicate with the first device 102. The second device 106 can also be a client type device as described for the first device 102. Another example, the first device 102 or the second device 106 can be a particularized machine, such as a portable computing device, a thin client, a notebook, a netbook, a smartphone, a tablet, a personal digital assistant, or a cellular phone, and as specific examples, an Apple iPhone™, Android™ smartphone, or Windows™ platform smartphone.

For illustrative purposes, the computing system 100 is described with the second device 106 as a non-mobile computing device, although it is understood that the second device 106 can be different types of computing devices. For example, the second device 106 can also be a mobile computing device, such as notebook computer, another client device, or a different type of client device. The second device 106 can be a standalone device, or can be incorporated with a vehicle, for example a car, truck, bus, or train.

Also for illustrative purposes, the computing system 100 is shown with the second device 106 and the first device 102 as end points of the communication path 104, although it is understood that the computing system 100 can have a different partition between the first device 102, the second device 106, and the communication path 104. For example, the first device 102, the second device 106, or a combination thereof can also function as part of the communication path 104.

The communication path 104 can be a variety of networks. For example, the communication path 104 can include wireless communication, wired communication, optical, ultrasonic, or the combination thereof. Satellite communication, cellular communication, Bluetooth, Infrared Data Association standard (IrDA), wireless fidelity (WiFi), and worldwide interoperability for microwave access (WiMAX) are examples of wireless communication that can be included in the communication path 104. Ethernet, digital subscriber line (DSL), fiber to the home (FTTH), and plain old telephone service (POTS) are examples of wired communication that can be included in the communication path 104.

Further, the communication path 104 can traverse a number of network topologies and distances. For example, the communication path 104 can include direct connection, personal area network (PAN), local area network (LAN), metropolitan area network (MAN), wide area network (WAN) or any combination thereof.

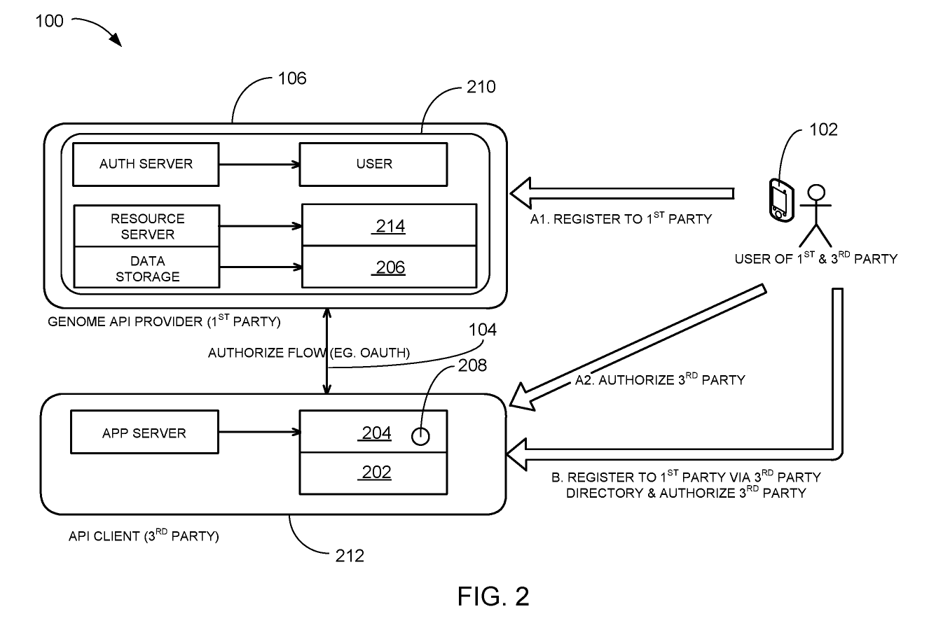

Referring now to FIG. 2, there is shown a first example of a registration process for the computing system 100. For clarity and brevity, the discussion of the embodiment of the present invention will focus on the first device 102 delivering the result generated by the computing system 100. However, the second device 106 and the first device 102 can be discussed interchangeably. The first device 102 and the second device 106 can communicate via the communication path 104. Genome information and genomic information are used interchangeably representing the same element.

A user profile 202 is defined as a compilation of information regarding a user of the computing system 100. For example, the user profile 202 can include user information 204, a user's ethnicity, a user's sex, a user's age, a user's genome information represented as a genomic raw data 206, or a combination thereof. The user information 204 is defined as information required to access the computing system 100. For example, the user information 204 can include a user identification 208, a password, an email address, or a combination thereof. The user identification 208 can represent a login identification. The user identification 208 can represent the email address or user generated login name.

The genomic raw data 206 can represent genetic information. For example, the genomic raw data 206 can represent user's complete set of deoxyribonucleic acid (DNA) information. For further example, the genomic raw data 206 can represent the information regarding user's genetic material.

The user of the computing system 100 can register the user information 204 directly to a provider 210 or via a third party 212. The provider 210 can represent an entity that provides a service or a platform of the computing system 100. For example, the provider 210 can provide the genome application programming interface (API) platform to the user of the first device 102, the third party 212, or a combination thereof. The genome API can represent the genomic information access mechanism provided by the computing system 100. The genome API platform can represent the computing system 100 to allow the user to access the user's genetic information from the first device 102, the second device 106, or a combination thereof. The third party 212 can represent an entity that provides the API client to access the computing system 100. For example, the API client can represent an app or software on the first device 102 created by the third party 212 to access the genome API of the provider 210.

The user can register for the service provided by the provider 210 by registering the user information 204, the genomic raw data 206, or a combination thereof. The provider 210 can create the user profile 202 including the user information 204, the genomic raw data 206, or a combination thereof. The provider 210 can store the user profile 202. As discussed below, the provider 210 can store a genome profile 214 including an interpretation of the genomic raw data 206. The genome profile 214 can represent a compilation of information related to the user's genetic information. Details regarding the genome profile 214 will be discussed below.

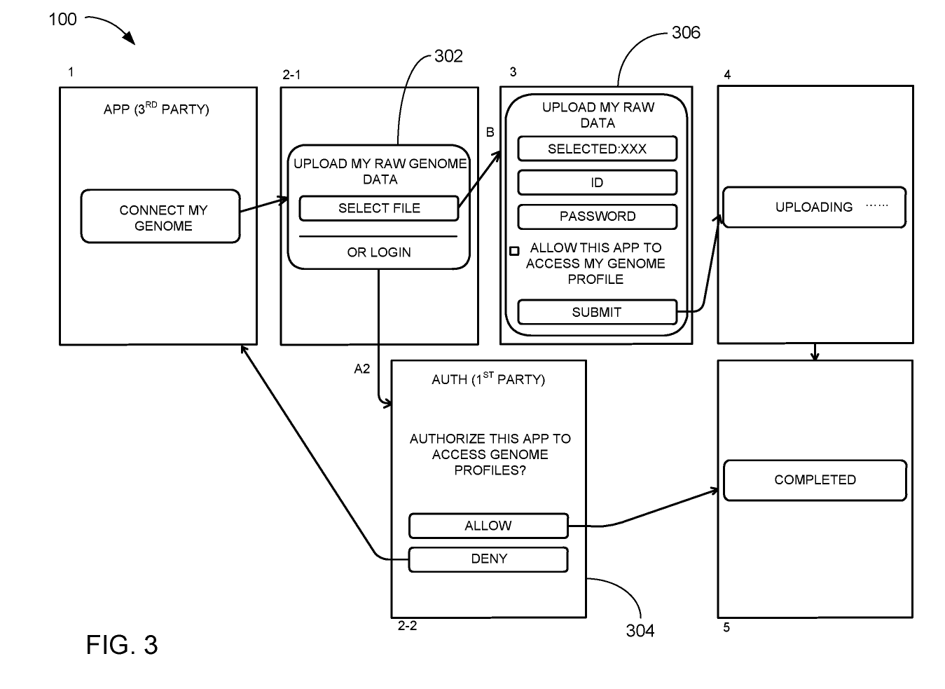

Referring now to FIG. 3, there is shown a second example of a registration process for the computing system 100. The computing system 100 can include various instances of an interface type 302 to register the user identification 208 of FIG. 2, the password, the genomic raw data 206 of FIG. 2, or a combination thereof. The interface type 302 can represent a classification of a user interface to access the computing system 100. For example, the interface type 302 can include a provider interface 304, a third party interface 306, or a combination thereof.

The provider interface 304 can represent a graphical user interface (GUI) of the provider 210 of FIG. 2 to access the computing system 100. The third party interface 306 can represent a GUI of the third party 212 of FIG. 2 to access the computing system 100. For example, FIG. 3 can represent the registration process via the third party interface 306 to register the user identification 208, the genomic raw data 206, or a combination thereof to the provider 210. The provider 210 can allow or deny whether the third party interface 306 can access the user information 204, the genomic raw data 206, or a combination thereof.

For further example, the user can use the DNA testing kit to provide the user's DNA information. A DNA sequencing service provider can generate the genomic raw data 206 based on the user's DNA information and return the genomic raw data 206 back to the user. The user can upload the genomic raw data 206 to the computing system 100 via the provider interface 304, the third party interface 306, or a combination thereof. Details regarding the upload are discussed below.

For a different example, the provider 210 can generate the genomic raw data 206 based on the user's DNA information provided via the DNA testing kit. More specifically as an example, the provider 210 or the DNA sequencing service provider can generate the genomic raw data 206 after receiving a request from the user. For this example, the uploading of the genomic raw data 206 is unnecessary as the provider 210 can store the genomic raw data 206 after generation.

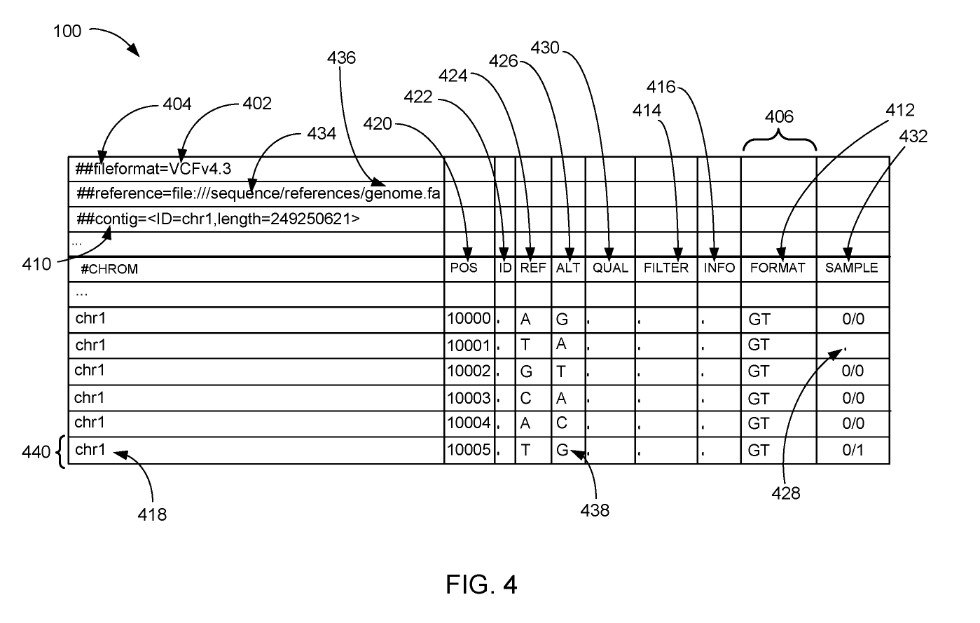

Referring now to FIG. 4, there is shown an example of the genomic raw data 206. The genomic raw data 206 can be represented by various instances of a sequencing result type 402. The sequencing result type 402 can represent a classification of result generated from a genetic sequencing process. For example, the sequencing result type 402 can include the whole genome sequencing (WGS), the whole exome sequencing (WES), the single nucleotide polymorphism (SNP) array, the targeted sequencing, or a combination thereof. The sequencing result type 402 can be represented in various instances of a file format 404. The file format 404 can represent a data structure, a file type, or a combination thereof. For example, the file format 404 of the genomic raw data 206 can include the Variant Call Format (VCF), the tab-separated values (tsv), the comma-separated values (csv), the Browser Extensible Data (BED) format, General Feature Format (GFF), genomic VCF (gVCF), SNP, or a combination thereof.

The file format 404 can include various instances of a genomic field 406. The genomic field 406 can represent a data field within the file format 404. For example, the file format 404 for the VCF can include the genomic field 406 different from the file format 404 represented in SNP. For further example, the genomic raw data 206 can include multiple instances of the genomic field 406.

The file format 404 of the VCF can include the genomic field 406 for the file format 404, a reference data 408, a contig data 410, a field format 412, a filter status 414, an additional information 416, or a combination thereof. For further example, the VCF can include the genomic field 406 for a chromosome data 418, a position data 420, a genome identification 422, a reference base (REF) data 424, an alternate base (ALT) data 426, a not available (NA) data 428, a genotype quality 430, a genotype sample 432, or a combination thereof.

As stated above, the file format 404 can represent the type of format used to organize the genomic raw data 206. For example, the file format 404 can represent VCFv.4.3. The reference data 408 can represent the information in the particular instance of the file format 404 to indicate which instance of a reference sequence 434 was used to analyze the genomic raw data 206. A reference sequence version 436 can represent the specific version of the reference sequence 434. The reference sequence 434 can represent a representative example of a species' set of genes. The reference sequence 434 can be represented in the file format 404 of FASTA, fai index, or a combination thereof. The genomic raw data 206 can include a variant data from the reference sequence 434 represented in VCF, tabix index, or a combination thereof. The contig data 410 can represent a set of overlapping DNA segments that together represent a consensus region of the DNA. For further example, the contig data 410 can represent the identification information for the chromosome. The field format 412 can include integer, float, character, string, or a combination thereof. The additional information 416 can also define the field format 412 based on values presented in the additional information 416. The additional information 416 can be used to encode structural variants. The filter status 414 can indicate whether the chromosome data 418 for the position data 420 passes filters or not.

The chromosome data 418 can represent a particular instance of the chromosome. For example, the chromosome data 418 can represent an identifier from the reference sequence 434. The genomic raw data 206 can be represented in the file format 404 of VCF. The chromosome data 418 can represent the identifier for the chromosome within the genomic raw data 206 in reference to the reference sequence 434. The position data 420 can represent a locus on a chromosome. For example, the position data 420 can represent a reference position relative to the reference sequence 434. More specifically as an example, the reference sequence 434 can include the position data 420 sorted numerically in increasing order, within each instance of the reference sequence 434 of the chromosome data 418. The position data 420 can include multiple instances of a genotype data 438. The genotype data 438 can include an allele. The allele can represent a viable DNA coding that occupies a given instance of the position data 420.

The REF data 424 can represent the allele in reference to the reference sequence 434. For example, the REF data 424 can represent the allele in reference to particular instance of the position data 420 of the reference sequence 434. The ALT data 426 can represent the allele that is variant from the reference sequence 434. For example, the ALT data 426 can represent a list of alternate non-reference alleles or the variant data. The NA data 428 can represent a result to indicate that the genotype data 438 was irretrievable. The genotype quality 430 can represent an accuracy score for the allele retrieved. The genotype sample 432 can represent a set of genes responsible for particular trait.

The genomic raw data 206 can include a genomic raw line 440. The genomic raw line 440 can represent each instance of the chromosome for the particular locus. For example, the genomic raw line 440 can include particular instance of the chromosome data 418 for particular instance of the position data 420. The genomic raw data 206 can include multiple instances of the genomic raw line 440.

The genome identification 422 can represent an identification information assigned to the genomic raw data 206 registered. For example, if the user registers the genomic raw data 206, the computing system 100 can assign the genome identification 422 for particular instance of the genomic raw data 206. For a different example, the user can register multiple different instances of the genomic raw data 206 including user's own instance of the genomic raw data 206 and the genomic raw data 206 for the user's kin. The computing system 100 can assign the same instance of the genome identification 422 for the multiple different instances of the genomic raw data 206. For further example, multiple users can register the same instance of the genomic raw data 206. The computing system 100 can assign the same instance of the genome identification 422 for the multiple users for that one instance of the genomic raw data 206.

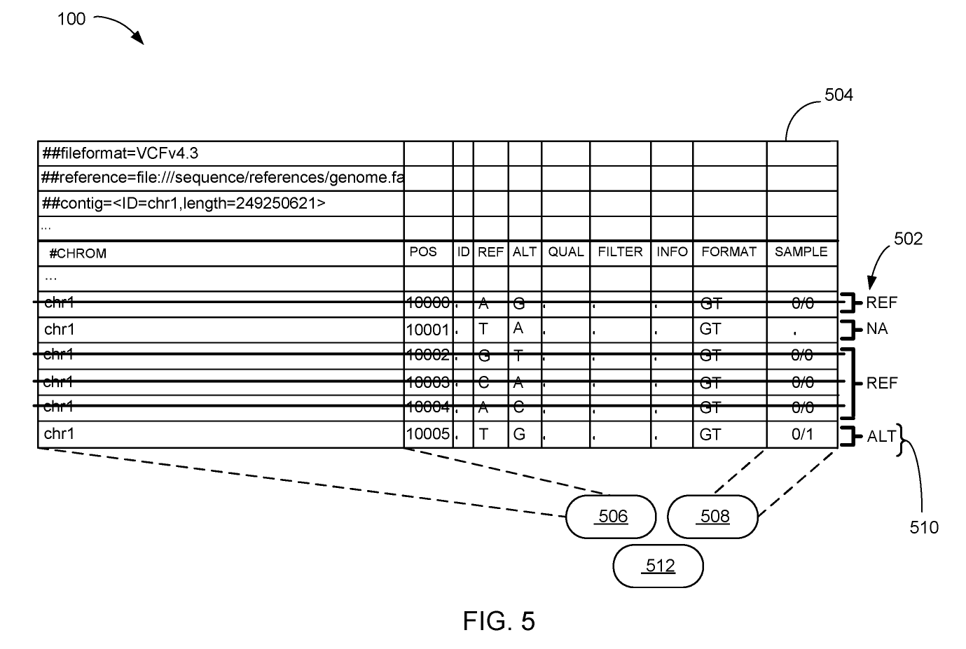

Referring now to FIG. 5, there is shown various examples of genomic information. A genomic reference line 502 can represent each instance of the chromosome for the particular locus for the reference sequence 434 of FIG. 4. For example, the genomic reference line 502 can include particular instance of the chromosome data 418 for particular instance of the position data 420. The reference sequence 434 can include multiple instances of the genomic reference line 502.

A conversion genomic data 504 can represent a processed instance of the genomic raw data 206 of FIG. 2. Once the genomic raw data 206 is processed by the computing system 100, the computing system 100 can convert the genomic raw data 206 as the conversion genomic data 504. The conversion genomic data 504 can include an abbreviated genomic data 506, a processed genomic data 508, or a combination thereof.

The abbreviated genomic data 506 can represent a filtered instance of the genomic raw data 206. The processed genomic data 508 can represent an unfiltered instance of the genomic raw data 206. For example, the abbreviated genomic data 506 can represent the genomic raw data 206 having the genomic raw line 440 that matches with the genomic reference line 502 of the reference sequence 434 removed. In contrast, the processed genomic data 508 can represent the genomic raw data 206 without the genomic raw line 440 being removed.

The processed genomic data 508, the abbreviated genomic data 506, or a combination thereof can include multiple instances of a converted genomic line 510. The converted genomic line 510 can represent the genomic raw line 440 that has the genotype quality 430 of FIG. 4 meeting or exceeding a quality threshold 512, that has been compared to the genomic reference line 502, or a combination thereof. The quality threshold 512 can represent a limit required for the genotype quality 430. For example, the quality threshold 512 can represent a minimum or maximum value for the genotype quality 430.

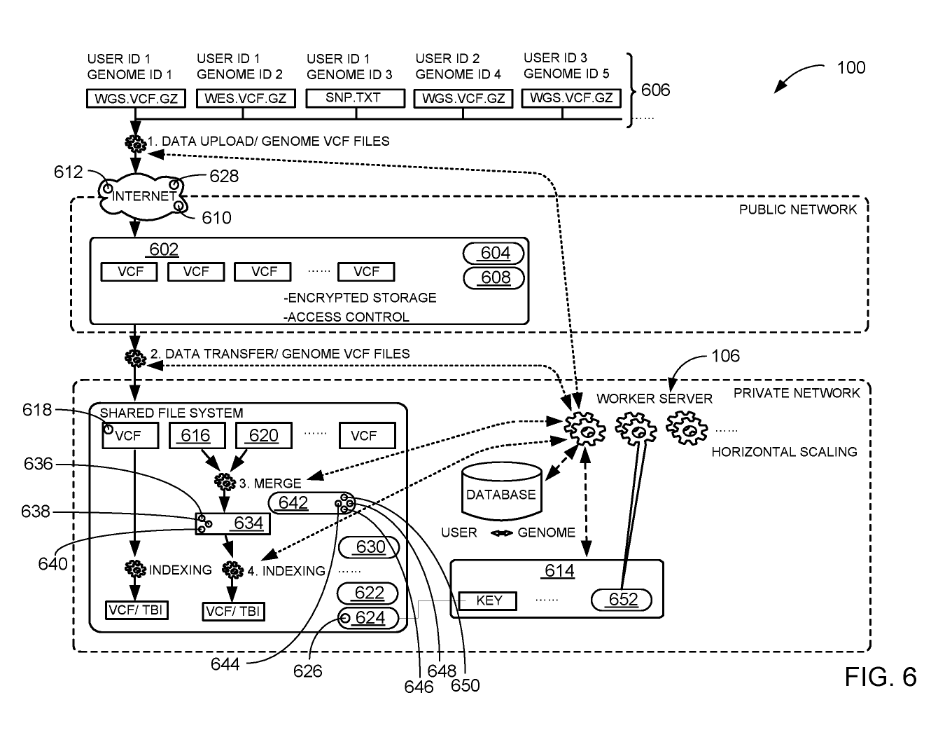

Referring now to FIG. 6, therein is shown an example of system architecture of the computing system 100. For example, the computing system 100 can utilize tabix/fai index to access VCF/FASTA file along with the second device 106 representing an application server, a worker server, or a combination thereof as a backend. For further example, the computing system 100 can store the genomic raw data 206 of FIG. 2 in a storage system 602. For a specific example, the storage system 602 can represent a network file system (NFS), shared file system, or a combination thereof. The storage system 602 can be mounted on the second device 106. Details regarding the storage system 602 are discussed below.

It has been discovered that mounting the storage system 602 representing the NFS on the second device 106 representing the application server allows the computing system 100 to store, encrypt, decrypt, index access, or a combination thereof the genomic raw data 206 by the application server. Traditionally, multiple instances of the application server are setup horizontally scaled for load balancing. An increase in one instance of the genomic raw data 206 increases the demand for storage space increase in the order of double digit gigabytes. As a result, adding the genomic raw data 206 leads further inefficiency to perform index searching or to rebuild index to search of the genomic raw data 206 due to a genomic data size 604 of the genomic raw data 206. Further, due to a large instance of the genomic data size 604 of the genomic raw data 206, the application server, without the NFS mounted, extracting, decompressing, accessing, or a combination thereof of the genomic raw data 206 is unrealistic due to performance degradation.

However, by mounting the NFS on the application server, the computing system 100 can increase the performance of the application server to extract, decompress, access, or a combination thereof of the genomic raw data 206. Moreover, as more instances of the genomic raw data 206 is handled by the computing system 100, by horizontally scaling multiple instances of the application server, the performance of the computing system 100 can be increased for further efficiency to handle numerous instances of the genomic raw data 206. By having the distributed architecture of horizontally scaling the second device 106 and mounting the storage system 602, the computing system 100 can improve the performance to process the genomic raw data 206 efficiently.

The computing system 100 can upload various instances of the genomic raw data 206, the conversion genomic data 504 of FIG. 5, or a combination thereof to the storage system 602. More specifically as an example, the computing system 100 can upload a user genomic file 606 to the storage system 602. The user genomic file 606 can represent a compilation data where the user information 204 of FIG. 2, the genome identification 422, or a combination thereof is correlated to the genomic raw data 206, the conversion genomic data 504, or a combination thereof.

The genomic data size 604 can represent a size measured in bits, bytes, or a combination thereof of the genomic information. For example, the genomic raw data 206 can be measured according to the genomic data size 604. A size threshold 608 can represent a limit on the genomic data size 604. For example, the size threshold 608 can represent the minimum or maximum data size required for the genomic data size 604.

A network speed 610 can represent a rate of data transfer. For example, the network speed 610 can represent how fast the data is transferred on the communication path 104 of FIG. 1. A speed threshold 612 can represent a limit on the network speed 610. For example, the speed threshold 612 can represent the minimum or the maximum speed required for the network speed 610. A key management system 614 can represent a device that manages and stores an encryption key. Details regarding the key management system 614 are discussed below.

A format consensus file 616 can represent the genomic information formatted into the file format 404 representing the VCF. For example, the format consensus file 616 can represent the genomic raw data 206, the conversion genomic data 504, or a combination thereof converted into the file format 404 representing the VCF. For example, the format consensus file 616 can include a VCF formatted line 618. The VCF formatted line 618 can represent the genomic raw line 440, the converted genomic line 510, or a combination thereof converted into the file format 404 representing the VCF. For example, the file format 404 for SNP array can be unstandardized. As a result, the computing system 100 can generate the format consensus file 616 according to the VCF instance of the file format 404 by including the chromosome data 418 of FIG. 4, the position data 420 of FIG. 4, the genotype data 438 of FIG. 4, the genotype sample 432 of FIG. 4, or a combination thereof.

A reference consensus file 620 can represent the genomic information converted into the file format 404 according to a system reference version 622. For example, the reference consensus file 620 can represent the genomic raw data 206, the conversion genomic data 504, or a combination thereof converted into the file format 404 representing the VCF according to the system reference version 622. The system reference version 622 can represent the reference sequence version 436 configured for the computing system 100. For example, the computing system 100 can compare the genomic raw data 206 to the reference sequence 434 having the version representing the system reference version 622. The reference sequence version 436 of the genomic raw data 206 can be different from the reference sequence version 436 or the system reference version 622 of the reference sequence 434.

A conversion table 624 can represent an arrangement of information including a conversion source version 626. The conversion source version 626 can represent the reference sequence version 436 that is convertible to the file format 404 specified according to the system reference version 622. For example, if the reference sequence version 436 of the genomic raw data 206 is included in the conversion table 624, the computing system 100 can convert the genomic raw data 206 into the reference consensus file 620 according to the system reference version 622. In contrast, if the reference sequence version 436 of the genomic raw data 206 is not included in the conversion table 624, the computing system 100 can generate a message 628 indicating an error that the conversion of the reference sequence version 436 is not supported.

A version difference 630 can represent a format difference between the reference sequence version 436 and the system reference version 622. For example, the file format 404 between the genomic raw data 206 based on the reference sequence version 436 can be different from the reference sequence 434 specified according to the system reference version 622. The version difference 630 can include the difference in the file format 404 due to different versions. A temporary file 632 can represent an interim file created by the computing system 100 to store information temporarily.

A unification genomic file 634 can represent a unified version of multiple genomic information of a one individual. For example, a user can upload multiple instances of the genomic raw data 206 to the computing system 100. For a specific example, one instance of the genomic raw data 206 can represent the sequencing result type 402 of FIG. 4 of WGS. And another instance of the genomic raw data 206 of the same individual can represent the sequencing result type 402 of SNP. The computing system 100 can unify the multiple instances of the genomic raw data 206 to generate the unification genomic file 634 for that one individual. For a different example, the computing system 100 can unify multiple instances of the genomic information formatted according to various instances of the file format 404 into the unification genomic file 634.

The unification genomic file 634 can include a unified genomic line 636. The unified genomic line 636 can represent each instance of the chromosome for the particular locus for the unification genomic file 634. A multi-sample file 638 can represent a genomic record including multiple instances of the genotype sample 432. For example, the computing system 100 can create the multi-sample file 638 based on a set of union sharing the same instance of the chromosome data 418 of FIG. 4, the position data 420 of FIG. 4, or a combination thereof. The multi-sample file 638 can include a multi-sample line 640. The multi-sample line 640 can represent each instance of the chromosome for the particular locus for the multi-sample file 638.

The computing system 100 can merge multiple instances of the genomic information based on a merge policy 642. The merge policy 642 can represent a condition on how to unify multiple instances of the genomic information. The merge policy 642 can include a majority vote policy 644, a conservative choice policy 646, an accuracy policy 648, a time period policy 650, or a combination thereof.

The majority vote policy 644 can represent a condition where the selection of the genotype sample 432 is based on majority number. For example, the number of the genotype sample 432 can represent three samples. Based on the majority vote policy 644, if there are at least two of the same samples of the genotype sample 432, the computing system 100 can select the genotype sample 432 with the same sample due to the majority number.

The conservative choice policy 646 can represent a condition where the non-selection of the genotype sample 432 is based on the existence of more than two different samples of the genotype sample 432. For example, if there are at least two different instances of the genotype sample 432, the computing system 100 can avoid selecting the genotype sample 432 due to inconsistency. The computing system 100 can instead determine the genotype sample 432 as the NA data 428 of FIG. 4.

The accuracy policy 648 can represent a condition where the selection of the genotype sample 432 is based on the highest instance of the genotype quality 430 of FIG. 4. The time period policy 650 can represent a condition where the selection of the genotype sample 432 is based on a time period 652 of when the genotype sample 432 is prepared. For example, the time period 652 can represent nanoseconds, microseconds, seconds, minutes, days, weeks, months, years, season, day, night, or a combination thereof.

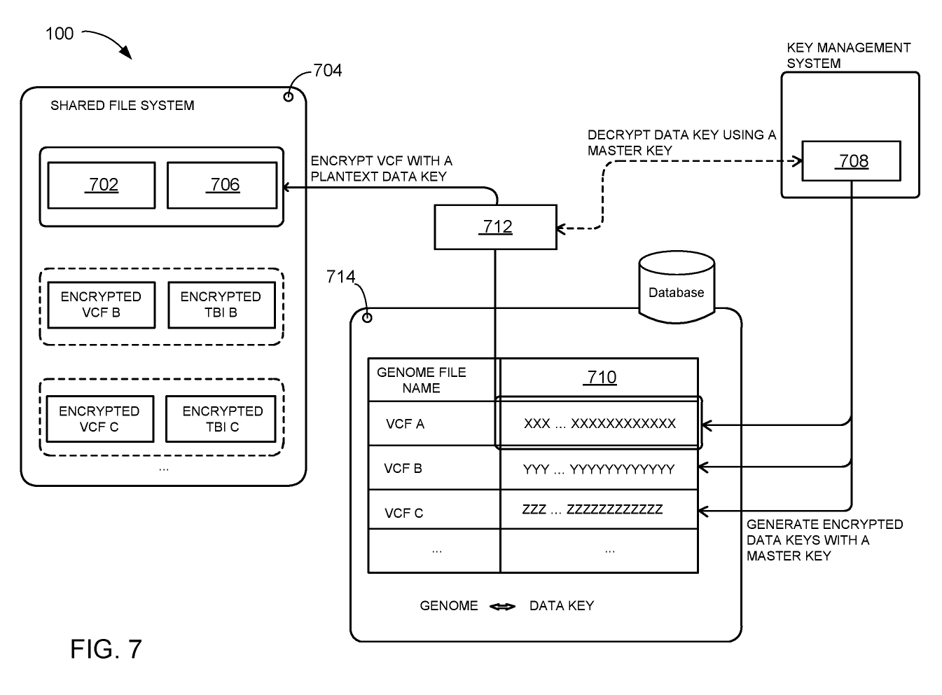

Referring now to FIG. 7, therein is shown an example of system architecture for encrypting the genomic information. An encrypted genomic data 702 can represent the genomic information that has been encrypted. For example, the computing system 100 can generate the encrypted genomic data 702 based on encrypting the conversion genomic data 504 of FIG. 5 according to an encryption type 704. The encryption type 704 can represent a classification of an encryption method. For example, the encryption type 704 can include a disk encryption, a file encryption, or a combination thereof.

An encrypted index 706 can represent encrypted instance of data that facilitates information retrieval by the computing system 100. For example, the encrypted index 706 can represent an encrypted tabix index. A master key 708 can represent data used to derive other encryption key(s). For example, the master key 708 can represent a symmetric master key used to derive other symmetric keys including data encryption keys, key wrapping keys, authentication keys, or a combination thereof using symmetric cryptographic methods. The key management system 614 of FIG. 6 can store the master key 708.

For further example, other keys can include an encrypted data key 710, a plain text data key 712, or a combination thereof. The encrypted data key 710 can represent a random string of bits created explicitly for scrambling and unscrambling data. The plain text data key 712 can represent a human readable form of the encrypted data key 710. A decrypted index 714 can represent a decrypted instance of the encrypted index 706. For example, the decrypted index 714 can represent a tabix index.

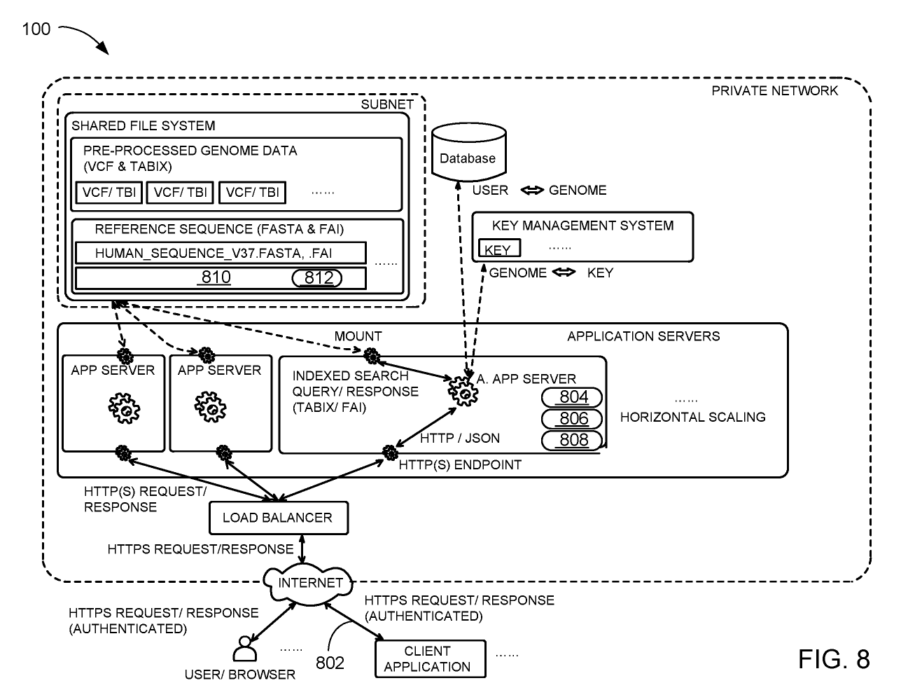

Referring now to FIG. 8, therein is shown an example of system architecture for retrieving the genomic information. For example, the computing system 100 can receive a user request 802 to retrieve a personal genomic data 804. The personal genomic data 804 can represent a user specified genomic information. For example, the user request 802 can specify the genomic information that the user wishes to retrieve. More specifically as an example, the user request 802 can include the genome identification 422 of FIG. 4, the chromosome data 418 of FIG. 4, the position data 420 of FIG. 4, or a combination thereof The position data 420 can include a start position 806, an end position 808, or a combination thereof. More specifically as an example, the user request 802 can include the start position 806, the end position 808, or a combination thereof to specify the range of genomic information that the user would like the computing system 100 to retrieve the user's genomic information.

A consensus sequence 810 can represent a calculated order of most frequent residues, either nucleotide or amino acid, found at each position in a sequence alignment. The sequence alignment can represent a way of arranging sequences of DNA, Ribonucleic acid (RNA), or protein to identify regions of similarity that may be a consequence of functional, structural, or evolutionary relationships between sequences. A sequence string 812 can represent a user specified range of the reference sequence 434. For example, the reference sequence 434 can present FASTA format file using fai index.

The second device 106 can represent the application server. The computing system 100 can include multiple instances of the application server horizontally scaled. When the application servers are booted, the storage system 602 representing the NFS can be mounted to the application servers. The NFS can include the variant data, the reference sequence 434, or a combination thereof.

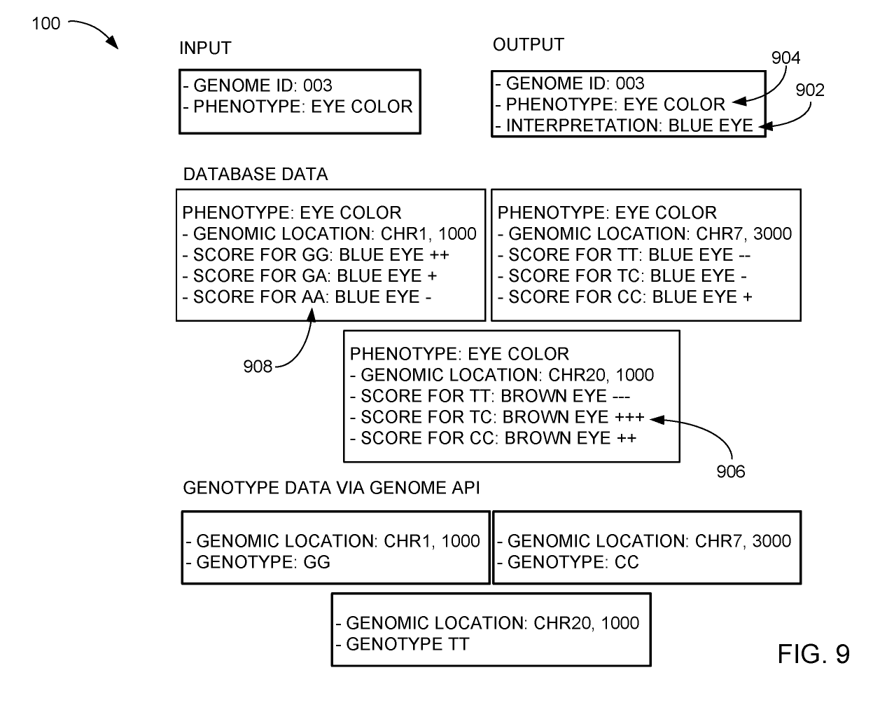

Referring now to FIG. 9, therein is shown an example of retrieving an interpretation data 902. The interpretation data 902 can represent an interpretation of a phenotype data 904. The phenotype data 904 can represent a composite of an organism's observable characteristic or trait. The phenotype data 904 can represent the physical expression, or characteristics, of the trait. For example, the phenotype data 904 can represent eye color. The interpretation data 902 for the genome identification 422 of FIG. 4 representing “003” for the phenotype data 904 of eye color can represent “Blue eye.”

A phenotype tendency 906 can represent a propensity for the phenotype data 904 to be interpreted as a specific instance of the interpretation data 902. For example, the computing system 100 can determine the phenotype tendency 906 based on a phenotype score 908. The phenotype score 908 can an alphanumeric value to grade the phenotype tendency 906. For example, the phenotype score 908 of “GG” can result in the interpretation data 902 of “Blue Eye++.”

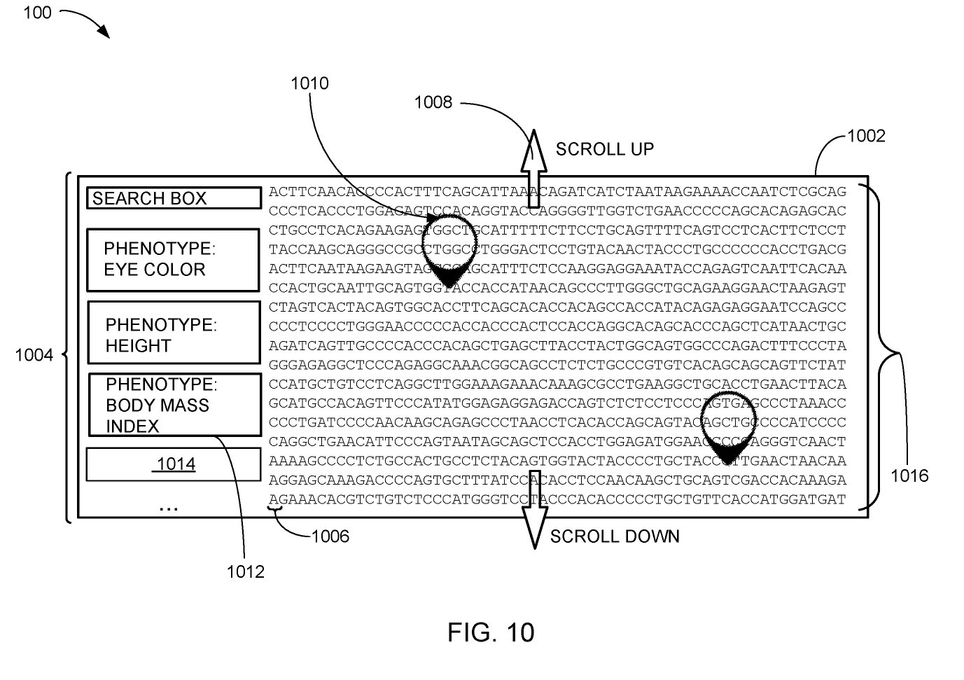

Referring now to FIG. 10, therein is shown an example of a display example of the personal genomic data 804 of FIG. 8. For example, the computing system 100 can display the personal genomic data 804 with a display interface 1002 of the first device 102 of FIG. 1. The display interface 1002 can represent a component of the first device 102 to display information to a user. For example, the display interface 1002 can represent a screen, a user interface, or a combination thereof.

More specifically as an example, the computing system 100 can change the display of the personal genomic data 804 according to a display size 1004 of the display interface 1002. The display size 1004 can represent a dimension of the display interface 1002. For example, the display size 1004 can represent a height, a width, or a combination thereof. A content size 1006 can represent a size of content. For example, the content size 1006 can represent a font size, a pixel size, or a combination thereof to display the personal genomic data 804. For further example, the user of the first device 102 can change the content size 1006 based on a user gesture 1008. The user gesture 1008 can represent an action performed on the first device 102. For example, the user gesture 1008 can include swipe, scroll, pinch, expand, shake, or a combination thereof.

The computing system 100 can display genome coordinates 1010. The genome coordinates 1010 can represent a position indicator for the personal genomic data 804. For example, the computing system 100 can indicate where in the personal genomic data 804 represents particular instance of the phenotype data 904 of FIG. 9 with the genome coordinates 1010.

A display format 1012 can represent a form to display the content. For example, the display format 1012 for the genome coordinates 1010 can represent a pin. For another example, the display format 1012 can include a display card, a list, or a combination thereof.

An associative research data 1014 can represent a research study associated to particular instance of the phenotype data 904. For example, the computing system 100 can display the associative research data 1014 for particular instance of the genome coordinates 1010 for the phenotype data 904 with the display format 1012 representing the display card.

A genomic portion 1016 can represent a subset of the personal genomic data 804. For example, the computing system 100 can display the genomic portion 1016 to limit the personal genomic data 804 that can be displayed on the display interface 1002.

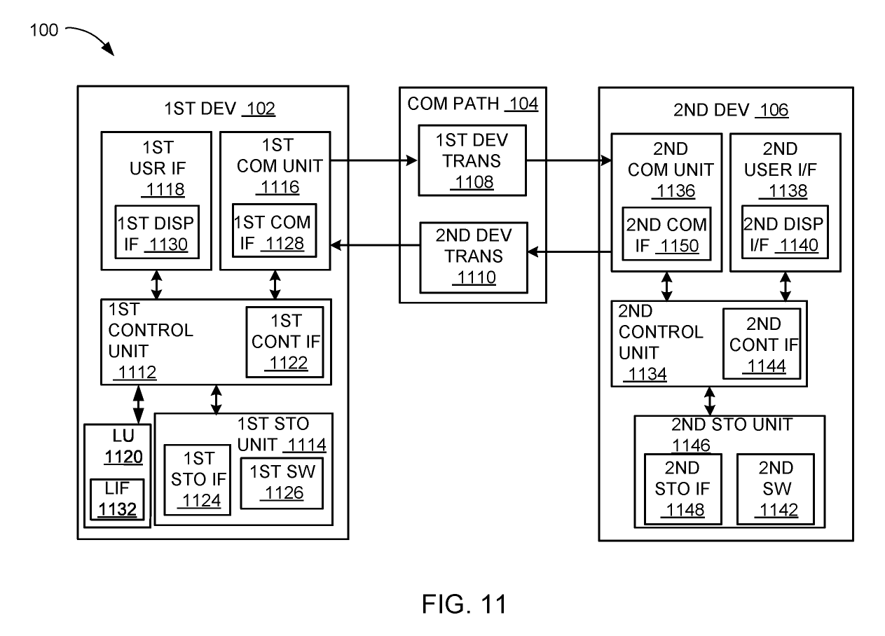

Referring now to FIG. 11, therein is shown an exemplary block diagram of the computing system 100. The computing system 100 can include the first device 102, the communication path 104, and the second device 106. The first device 102 can send information in a first device transmission 1108 over the communication path 104 to the second device 106. The second device 106 can send information in a second device transmission 1110 over the communication path 104 to the first device 102.

For illustrative purposes, the computing system 100 is shown with the first device 102 as a client device, although it is understood that the computing system 100 can have the first device 102 as a different type of device. For example, the first device 102 can be a server.

Also for illustrative purposes, the computing system 100 is shown with the second device 106 as a server, although it is understood that the computing system 100 can have the second device 106 as a different type of device. For example, the second device 106 can be a client device.

For brevity of description in this embodiment of the present invention, the first device 102 will be described as a client device and the second device 106 will be described as a server device. The present invention is not limited to this selection for the type of devices. The selection is an example of the present invention.

The first device 102 can include a first control unit 1112, a first storage unit 1114, a first communication unit 1116, a first user interface 1118, and a location unit 1120. The first control unit 1112 can include a first control interface 1122. The first control unit 1112 can execute a first software 1126 to provide the intelligence of the computing system 100. The first control unit 1112 can be implemented in a number of different manners. For example, the first control unit 1112 can be a processor, an embedded processor, a microprocessor, a hardware control logic, a hardware finite state machine (FSM), a digital signal processor (DSP), or a combination thereof. The first control interface 1122 can be used for communication between the first control unit 1112 and other functional units in the first device 102. The first control interface 1122 can also be used for communication that is external to the first device 102.

The first control interface 1122 can receive information from the other functional units or from external sources, or can transmit information to the other functional units or to external destinations. The external sources and the external destinations refer to sources and destinations physically separate from the first device 102.

The first control interface 1122 can be implemented in different ways and can include different implementations depending on which functional units or external units are being interfaced with the first control interface 1122. For example, the first control interface 1122 can be implemented with a pressure sensor, an inertial sensor, a microelectromechanical system (MEMS), optical circuitry, waveguides, wireless circuitry, wireline circuitry, or a combination thereof.

The location unit 1120 can generate location information, current heading, and current speed of the first device 102, as examples. The location unit 1120 can be implemented in many ways. For example, the location unit 1120 can function as at least a part of a global positioning system (GPS), an inertial computing system, a cellular-tower location system, a pressure location system, or any combination thereof.

The location unit 1120 can include a location interface 1132. The location interface 1132 can be used for communication between the location unit 1120 and other functional units in the first device 102. The location interface 1132 can also be used for communication that is external to the first device 102.

The location interface 1132 can receive information from the other functional units or from external sources, or can transmit information to the other functional units or to external destinations. The external sources and the external destinations refer to sources and destinations physically separate from the first device 102.

The location interface 1132 can include different implementations depending on which functional units or external units are being interfaced with the location unit 1120. The location interface 1132 can be implemented with technologies and techniques similar to the implementation of the first control interface 1122.

The first storage unit 1114 can store the first software 1126. The first storage unit 1114 can also store the relevant information, such as advertisements, points of interest (POI), navigation routing entries, or any combination thereof.

The first storage unit 1114 can be a volatile memory, a nonvolatile memory, an internal memory, an external memory, or a combination thereof. For example, the first storage unit 1114 can be a nonvolatile storage such as non-volatile random access memory (NVRAM), Flash memory, disk storage, or a volatile storage such as static random access memory (SRAM).

The first storage unit 1114 can include a first storage interface 1124. The first storage interface 1124 can be used for communication between the location unit 1120 and other functional units in the first device 102. The first storage interface 1124 can also be used for communication that is external to the first device 102.

The first storage interface 1124 can receive information from the other functional units or from external sources, or can transmit information to the other functional units or to external destinations. The external sources and the external destinations refer to sources and destinations physically separate from the first device 102.

The first storage interface 1124 can include different implementations depending on which functional units or external units are being interfaced with the first storage unit 1114. The first storage interface 1124 can be implemented with technologies and techniques similar to the implementation of the first control interface 1122.

The first communication unit 1116 can enable external communication to and from the first device 102. For example, the first communication unit 1116 can permit the first device 102 to communicate with the second device 106, an attachment, such as a peripheral device or a computer desktop, and the communication path 104.

The first communication unit 1116 can also function as a communication hub allowing the first device 102 to function as part of the communication path 104 and not limited to be an end point or terminal unit to the communication path 104. The first communication unit 1116 can include active and passive components, such as microelectronics or an antenna, for interaction with the communication path 104.

The first communication unit 1116 can include a first communication interface 1128. The first communication interface 1128 can be used for communication between the first communication unit 1116 and other functional units in the first device 102. The first communication interface 1128 can receive information from the other functional units or can transmit information to the other functional units.

The first communication interface 1128 can include different implementations depending on which functional units are being interfaced with the first communication unit 1116. The first communication interface 1128 can be implemented with technologies and techniques similar to the implementation of the first control interface 1122.

The first user interface 1118 allows a user (not shown) to interface and interact with the first device 102. The first user interface 1118 can include an input device and an output device. Examples of the input device of the first user interface 1118 can include a keypad, a touchpad, soft-keys, a keyboard, a microphone, a camera, or any combination thereof to provide data and communication inputs.

The first user interface 1118 can include a first display interface 1130. The first display interface 1130 can include a display, a projector, a video screen, a speaker, a headset, or any combination thereof.

The first control unit 1112 can operate the first user interface 1118 to display information generated by the computing system 100. The first control unit 1112 can also execute the first software 1126 for the other functions of the computing system 100, including receiving location information from the location unit 1120. The first control unit 1112 can further execute the first software 1126 for interaction with the communication path 104 via the first communication unit 1116.

The second device 106 can be optimized for implementing the present invention in a multiple device embodiment with the first device 102. The second device 106 can provide the additional or higher performance processing power compared to the first device 102. The second device 106 can include a second control unit 1134, a second communication unit 1136, and a second user interface 1138.

The second user interface 1138 allows a user (not shown) to interface and interact with the second device 106. The second user interface 1138 can include an input device and an output device. Examples of the input device of the second user interface 1138 can include a keypad, a touchpad, soft-keys, a keyboard, a microphone, a camera, or any combination thereof to provide data and communication inputs. Examples of the output device of the second user interface 1138 can include a second display interface 1140. The second display interface 1140 can include a display, a projector, a video screen, a speaker, a headset, or any combination thereof.

The second control unit 1134 can execute a second software 1142 to provide the intelligence of the second device 106 of the computing system 100. The second software 1142 can operate in conjunction with the first software 1126. The second control unit 1134 can provide additional performance compared to the first control unit 1112.

The second control unit 1134 can operate the second user interface 1138 to display information. The second control unit 1134 can also execute the second software 1142 for the other functions of the computing system 100, including operating the second communication unit 1136 to communicate with the first device 102 over the communication path 104.

The second control unit 1134 can be implemented in a number of different manners. For example, the second control unit 1134 can be a processor, an embedded processor, a microprocessor, a hardware control logic, a hardware finite state machine (FSM), a digital signal processor (DSP), or a combination thereof.

The second control unit 1134 can include a second control interface 1144. The second control interface 1144 can be used for communication between the second control unit 1134 and other functional units in the second device 106. The second control interface 1144 can also be used for communication that is external to the second device 106.

The second control interface 1144 can receive information from the other functional units or from external sources, or can transmit information to the other functional units or to external destinations. The external sources and the external destinations refer to sources and destinations physically separate from the second device 106.

The second control interface 1144 can be implemented in different ways and can include different implementations depending on which functional units or external units are being interfaced with the second control interface 1144. For example, the second control interface 1144 can be implemented with a pressure sensor, an inertial sensor, a microelectromechanical system (MEMS), optical circuitry, waveguides, wireless circuitry, wireline circuitry, or a combination thereof.

A second storage unit 1146 can store the second software 1142. The second storage unit 1146 can also store the relevant information, such as advertisements, points of interest (POI), navigation routing entries, or any combination thereof. The second storage unit 1146 can be sized to provide the additional storage capacity to supplement the first storage unit 1114.

For illustrative purposes, the second storage unit 1146 is shown as a single element, although it is understood that the second storage unit 1146 can be a distribution of storage elements. Also for illustrative purposes, the computing system 100 is shown with the second storage unit 1146 as a single hierarchy storage system, although it is understood that the computing system 100 can have the second storage unit 1146 in a different configuration. For example, the second storage unit 1146 can be formed with different storage technologies forming a memory hierarchal system including different levels of caching, main memory, rotating media, or off-line storage.

The second storage unit 1146 can be a volatile memory, a nonvolatile memory, an internal memory, an external memory, or a combination thereof. For example, the second storage unit 1146 can be a nonvolatile storage such as non-volatile random access memory (NVRAM), Flash memory, disk storage, or a volatile storage such as static random access memory (SRAM).

The second storage unit 1146 can include a second storage interface 1148. The second storage interface 1148 can be used for communication between the location unit 1120 and other functional units in the second device 106. The second storage interface 1148 can also be used for communication that is external to the second device 106.

The second storage interface 1148 can receive information from the other functional units or from external sources, or can transmit information to the other functional units or to external destinations. The external sources and the external destinations refer to sources and destinations physically separate from the second device 106.

The second storage interface 1148 can include different implementations depending on which functional units or external units are being interfaced with the second storage unit 1146. The second storage interface 1148 can be implemented with technologies and techniques similar to the implementation of the second control interface 1144.

The second communication unit 1136 can enable external communication to and from the second device 106. For example, the second communication unit 1136 can permit the second device 106 to communicate with the first device 102 over the communication path 104.

The second communication unit 1136 can also function as a communication hub allowing the second device 106 to function as part of the communication path 104 and not limited to be an end point or terminal unit to the communication path 104. The second communication unit 1136 can include active and passive components, such as microelectronics or an antenna, for interaction with the communication path 104.

The second communication unit 1136 can include a second communication interface 1150. The second communication interface 1150 can be used for communication between the second communication unit 1136 and other functional units in the second device 106. The second communication interface 1150 can receive information from the other functional units or can transmit information to the other functional units.

The second communication interface 1150 can include different implementations depending on which functional units are being interfaced with the second communication unit 1136. The second communication interface 1150 can be implemented with technologies and techniques similar to the implementation of the second control interface 1144.

The first communication unit 1116 can couple with the communication path 104 to send information to the second device 106 in the first device transmission 1108. The second device 106 can receive information in the second communication unit 1136 from the first device transmission 1108 of the communication path 104.

The second communication unit 1136 can couple with the communication path 104 to send information to the first device 102 in the second device transmission 1110. The first device 102 can receive information in the first communication unit 1116 from the second device transmission 1110 of the communication path 104. The computing system 100 can be executed by the first control unit 1112, the second control unit 1134, or a combination thereof.

For illustrative purposes, the second device 106 is shown with the partition having the second user interface 1138, the second storage unit 1146, the second control unit 1134, and the second communication unit 1136, although it is understood that the second device 106 can have a different partition. For example, the second software 1142 can be partitioned differently such that some or all of its function can be in the second control unit 1134 and the second communication unit 1136. Also, the second device 106 can include other functional units not shown in FIG. 11 for clarity.

The functional units in the first device 102 can work individually and independently of the other functional units. The first device 102 can work individually and independently from the second device 106 and the communication path 104.

The functional units in the second device 106 can work individually and independently of the other functional units. The second device 106 can work individually and independently from the first device 102 and the communication path 104.

For illustrative purposes, the computing system 100 is described by operation of the first device 102 and the second device 106. It is understood that the first device 102 and the second device 106 can operate any of the modules and functions of the computing system 100. For example, the first device 102 is described to operate the location unit 1120, although it is understood that the second device 106 can also operate the location unit 1120.

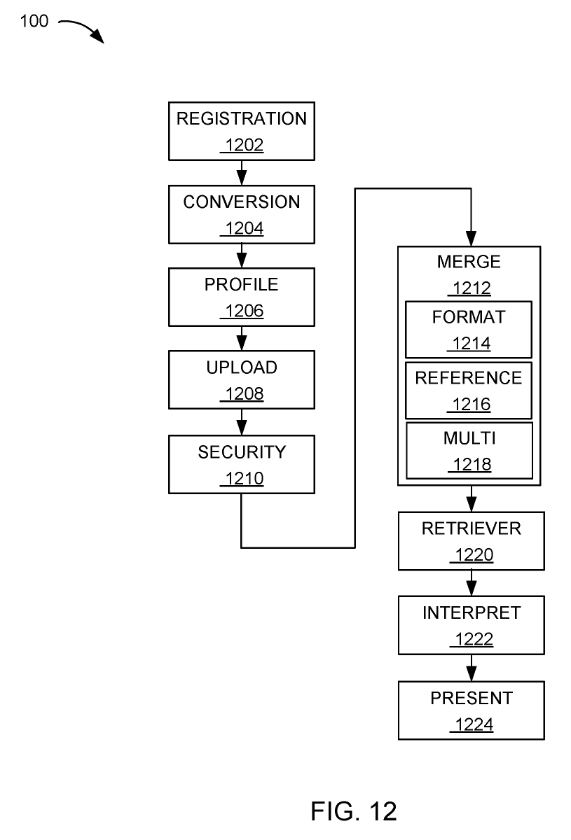

Referring now to FIG. 12, therein is shown a control flow of the computing system 100. The computing system can include a registration module 1202. The registration module 1202 registers the user information 204. For example, the computing system 100 can register the user information 204 including the user profile 202 of FIG. 2, the genomic raw data 206 of FIG. 2, or a combination thereof.

The registration module 1202 can register the user information 204 in a number of ways. For example, the user of the computing system 100 can register the user information 204 via the provider 210 of FIG. 2, the third party 212 of FIG. 2, or a combination thereof. More specifically as an example, the registration module 1202 can register the user information 204 based on the interface type 302 of FIG. 3. For a specific example, the interface type 302 can include the provider interface 304 of FIG. 3, the third party interface 306 of FIG. 3, or a combination thereof.

The registration module 1202 can register the user information 204 via the provider interface 304, the third party interface 306, or a combination thereof. The provider interface 304 and the third party interface 306 can be different from one another. If the user directly registers the user information 204 with the provider 210, the registration module 1202 can register the user information 204 via the provider interface 304.

For a different example, the user can use the third party interface 306 for the registration module 1202 to register the user information 204. More specifically as an example, the third party 212 can interact with the provider 210 based on the authorization provided by the provider 210. The authorization can represent OAuth. Via the third party interface 306 representing the application programming interface (API) client, the app, the software, or a combination thereof of the third party 212 can receive authorization from the provider 210 for the registration module 1202 to register the user information 204. The third party interface 306 can provide a form to fill out the user information 204 to be validated by the provider 210 to register the user information 204.

For example, the registration module 1202 can register the user information 204 including the user identification 208 of FIG. 2, the password, email address, or a combination thereof to be stored by the provider 210. For further example, the registration module 1202 can register the genomic raw data 206 selected by the user to be stored by the provider 210. The genomic raw data 206 can include the sequencing result type 402 of FIG. 4. For example, the sequencing result type 402 can include the whole genome sequencing (WGS), the whole exome sequencing (WES), the single nucleotide polymorphism (SNP) array, or a combination thereof.

The sequencing result type 402 can be represented in various types of the file format 404 of FIG. 4. For example, WGS, WES, or a combination thereof can be represented in the file format 404 representing the Variant Call Format (VCF) and SNP can be represented in the text format. More specifically as an example, one text format for SNP can be different from another text format for the SNP, resulting in variations of text format between one SNP to another. The file format 404 can include the Browser Extensible Data (BED) format, General Feature Format (GFF), genomic VCF (gVCF), or a combination thereof. The registration module 1202 can transmit the user information 204 to a conversion module 1204.

The computing system 100 can include the conversion module 1204, which can be coupled to the registration module 1202. The conversion module 1204 generates the conversion genomic data 504 of FIG. 5. For example, the conversion module 1204 can generate the conversion genomic data 504 based on the genomic raw data 206, the reference sequence 434 of FIG. 4, the quality threshold 512 of FIG. 5, the genomic data size 604 of FIG. 6, the size threshold 608 of FIG. 6, the network speed 610 of FIG. 6, the speed threshold 612 of FIG. 6, the sequencing result type 402, or a combination thereof. Details regarding the conversion module 1204 are discussed below. The conversion module 1204 can transmit the conversion genomic data 504 to a profile module 1206.

The computing system 100 can include the profile module 1206, which can be coupled to the conversion module 1204. The profile module 1206 generates the user genomic file 606 of FIG. 6. For example, the profile module 1206 can generate the user genomic file 606 based on the conversion genomic data 504, the user information 204, or a combination thereof.

The profile module 1206 can generate the user genomic file 606 in a number of ways. For example, the profile module 1206 can generate the user genomic file 606 by tying the user information 204 to the conversion genomic data 504. More specifically as an example, the user information 204 can include the genomic raw data 206. The conversion genomic data 504 can be generated from the genomic raw data 206. The profile module 1206 can correlate the user information 204 to the genomic raw data 206 converted as represented in the conversion genomic data 504.

For further example, the profile module 1206 can generate the genome identification 422 of FIG. 4 for each of the conversion genomic data 504. The profile module 1206 can correlate the user information 204 including the user identification 208 to each instance of the genome identification 422. More specifically as an example, one user having the user identification 208 can have multiple instances of the conversion genomic data 504, thus, having multiple instances of the genome identification 422 for each of the conversion genomic data 504. The profile module 1206 can generate the user genomic file 606 including the user identification 208 having the genome identification 422 assigned to the conversion genomic data 504. The profile module 1206 can transmit the user genomic file 606 to an upload module 1208.

The computing system 100 can include the upload module 1208, which can be coupled to the profile module 1206. The upload module 1208 uploads the user genomic file 606. For example, the upload module 1208 can upload the user genomic file 606 based on the interface type 302.

For a specific example, the user can upload the user genomic file 606 via the provider interface 304, the third party interface 306, or a combination thereof. As discussed above, if the user registers with the provider 210 via the provider interface 304 and selects the genomic raw data 206 to upload, the upload module 1208 can upload the user genomic file 606 to the storage system 602 of FIG. 6 of the second device 106 of FIG. 1. The storage system 602 can include the first storage unit 1114 of FIG. 11, the second storage unit 1146 of FIG. 11, or a combination thereof as discussed above.

For a different example, if the user registers with the provider 210 via the third party interface 306 and selects the genomic raw data 206 to upload, the upload module 1208 can upload the user genomic file 606 to the second device 106 from the API client, the app, the software, or a combination thereof of the third party 212. The upload module 1208 can transmit the user genomic file 606 to a security module 1210.

The computing system 100 can include the security module 1210, which can be coupled to the upload module 1208. The security module 1210 generates the encrypted genomic data 702 of FIG. 7. For example, the security module 1210 can encrypt the conversion genomic data 504 to generate the encrypted genomic data 702 based on the encryption type 704 of FIG. 7, the storage system 602, or a combination thereof.

The security module 1210 can generate the encrypted genomic data 702 in a number of ways. For example, the security module 1210 can generate the encrypted genomic data 702 based on the encryption type 704 representing the disk encryption of the storage system 602 in the second device 106 representing the web server, cloud computing resource, or a combination thereof. More specifically as an example, the security module 1210 can encrypt the entire instance of the storage system 602 storing the conversion genomic data 504 to generate the encrypted genomic data 702.

For further example, the security module 1210 can encrypt the storage system 602 of the second device 106 within the communication path 104 of FIG. 1 representing the public network. The security module 1210 can transfer the encrypted genomic data 702 from the storage system 602 in the public network to another different instance of the storage system 602 of the second device 106 within the communication path 104 representing the private network. More specifically as an example, the security module 1210 can decrypt the storage system 602 to convert the encrypted genomic data 702 back to the conversion genomic data 504 prior to mounting the conversion genomic data 504 on the storage system 602 within the private network.

For a different example, the security module 1210 can generate the encrypted genomic data 702 based on the encryption type 704 representing the file encryption to the storage system 602 representing the network file system (NFS) of the second device 106. The second device 106 with the NFS can be within the private network. More specifically as an example, the security module 1210 can encrypt the conversion genomic data 504 on per file basis rather than the entire instance of the storage system 602.

For a specific example, the security module 1210 can generate the encrypted genomic data 702 based on BGZF block-level encryption. Moreover, the security module 1210 can encrypt the conversion genomic data 504 based on the BGZF encryption via Advanced Encryption Standard (AES)-256 encryption. More specifically as an example, by encrypting based on BGZF with AES-256, the encrypted genomic data 702 can be organized in multiple blocks in sequential order. For a specific example, each block of the encrypted genomic data 702 can include the encrypted BGZF header, Secure Hash Algorithm 2 (SHA-2) key, and compressed and encrypted instance of the conversion genomic data 504. The encrypted genomic data 702 can include multiple blocks. By using the BGZF encryption, the security module 1210 can encrypt the conversion genomic data 504 compressed under BGZF and generate the encrypted index 706 of FIG. 7.

The computing system 100 can include the key management system 614 of FIG. 6. The key management system 614 can store the master key 708 of FIG. 7. The security module 1210 can generate the encrypted data key 710 of FIG. 7 and the plain text data key 712 of FIG. 7 for each of the conversion genomic data 504 to be encrypted based on the master key 708. The encrypted data key 710 and the plain text data key 712 are mapped to each other. The security module 1210 can generate the encrypted genomic data 702 based on using the plain text data key 712 and perform the BGZF compression to encrypt the conversion genomic data 504.

Continuing with the example, the security module 1210 can generate the encrypted index 706 to locate the conversion genomic data 504 within the storage system 602. The encrypted index 706 can represent the encrypted tabix index. More specifically as an example, the security module 1210 can generate the encrypted index 706 based on the plain text data key 712. The security module 1210 can store the encrypted data key 710 within the storage system 602. The security module 1210 can delete the plain text data key 712.

For further example, the security module 1210 can generate the conversion genomic data 504 based on decrypting the encrypted genomic data 702 on per file basis. More specifically as an example, the security module 1210 can retrieve the encrypted data key 710 from the storage system 602. The security module 1210 can decrypt the encrypted data key 710 by designating the master key 708, create the plain text data key 712, or a combination thereof. The security module 1210 can generate the decrypted index 714 of FIG. 7 based on the plain text data key 712, the encrypted index 706, or a combination thereof. The security module 1210 can perform the index search on the encrypted genomic data 702 with the decrypted index 714 of tabix index to decrypt the encrypted genomic data 702 that the search hits. The security module 1210 can generate the conversion genomic data 504 in the file format 404 including the VCF by decrypting the encrypted genomic data 702 which the index search hits on the decrypted index 714. The security module 1210 can delete the plain text data key 712. The security module 1210 can transmit the encrypted genomic data 702, the conversion genomic data 504, or a combination thereof to a merge module 1212.

The computing system 100 can include the merge module 1212, which can be coupled to the security module 1210. The merge module 1212 generates the various types of genome file. For example, the merge module 1212 can generate the format consensus file 616 of FIG. 6, the reference consensus file 620 of FIG. 6, the unification genomic file 634 of FIG. 6, or a combination thereof.

The merge module 1212 can generate the various types of genome file in a number of ways. For example, the merge module 1212 can decrypt the encrypted genomic data 702 similarly as the security module 1210 decrypting the encrypted genomic data 702 as discussed above. Based on the BGZF encryption format, the merge module 1212 can decrypt the encrypted genomic data 702 one block at a time in sequential order. More specifically as an example, the merge module 1212 can decrypt the encrypted genomic data 702 partially and not decrypting the entire instance of the encrypted genomic data 702. The merge module 1212 can generate the conversion genomic data 504 based on decrypting the encrypted genomic data 702.

The merge module 1212 can include a format module 1214. The format module 1214 generates the format consensus file 616. For example, the format module 1214 can generate the format consensus file 616 including the VCF formatted line 618 of FIG. 6 based on the conversion genomic data 504, the file format 404, the genomic field 406, or a combination thereof. For a specific example, the format module 1214 can generate the format consensus file 616 by converting the conversion genomic data 504 with the file format 404 other than the VCF into the file format 404 representing VCF. Details regarding format module 1214 are discussed below. The format module 1214 can transmit the format consensus file 616 to a reference module 1216.

The merge module 1212 can include the reference module 1216, which can be coupled to the format module 1214. The reference module 1216 generates the reference consensus file 620. For example, the reference module 1216 can determine whether the reference sequence version 436 of FIG. 4 for the conversion genomic data 504, the format consensus file 616, or a combination thereof matches with the system reference version 622 of FIG. 6. Details regarding the reference module 1216 are discussed below. The reference module 1216 can transmit the reference consensus file 620 to a multi module 1218.

The merge module 1212 can include the multi module 1218, which can be coupled to the reference module 1216. The multi module 1218 generates the unification genomic file 634. For example, the multi module 1218 can generate the unification genomic file 634 based on the conversion genomic data 504, the format consensus file 616, the reference consensus file 620, or a combination thereof. Details regarding the multi module 1218 are discussed below.

The merge module 1212 can encrypt the unification genomic file 634 similarly as the security module 1210 generating the encrypted genomic data 702 as discussed above. More specifically as an example, the merge module 1212 can encrypt the unification genomic file 634 based on the BGZF encryption via Advanced Encryption Standard (AES)-256 encryption.

For further example, the merge module 1212 can generate the encrypted index 706 to locate the unification genomic file 634 similarly as the security module 1210 generating the encrypted index 706 to locate the conversion genomic data 504. The encrypted index 706 can represent the encrypted tabix index. For further example, the multi module 1218 can generate the unification genomic file 634, generate the encrypted index 706, or a combination thereof under horizontal scaling architecture by multiple different instances of the second device 106 to load balance the computing resource. The merge module 1212 can transmit the unification genomic file 634, the encrypted index 706, or a combination thereof to a retriever module 1220.

The computing system 100 can include the retriever module 1220, which can be coupled to the merge module 1212. The retriever module 1220 retrieves the personal genomic data 804 of FIG. 8. For example, the retriever module 1220 can retrieve the personal genomic data 804 including the genotype data 438, the consensus sequence 810 of FIG. 8, or a combination thereof based on the unification genomic file 634, the user request 802 of FIG. 8, the encrypted index 706, or a combination thereof. Details regarding the retriever module 1220 are discussed below. The retriever module 1220 can transmit the personal genomic data 804 to an interpretation module 1222.

The computing system 100 can include the interpretation module 1222, which can be coupled to the retriever module 1220. The interpretation module 1222 generates the interpretation data 902 of FIG. 9. For example, the interpretation module 1222 can generate the interpretation data 902 based on the personal genomic data 804, the user request 802, or a combination thereof.

The interpretation module 1222 can generate the interpretation data 902 in a number of ways. For example, the interpretation module 1222 can retrieve the phenotype score 908 of FIG. 9 indicating the phenotype tendency 906 of FIG. 9 for each of the genotype data 438 for the position data 420 from the storage system 602. For further example, the storage system 602 can store the phenotype score 908 for each of the genotype data 438. Further, the interpretation module 1222 can retrieve the genotype data 438 for the position data 420 using the application programming interface (API) including the genome API.

For a specific example, the storage system 602 can include the position data 420 representing “1000” for the chromosome data 418 representing “chr1.” The phenotype score 908 for the genotype data 438 representing “GG” can be “Blue Eye++” for the phenotype data 904 of FIG. 9 representing “eye color.” For another example, the phenotype score 908 for the genotype data 438 representing “GA” can be “Blue Eye+” for the phenotype data 904 representing “eye color.” For different example, the phenotype score 908 for the genotype data 438 representing “AA” can be “Blue Eye −” for the phenotype data 904 representing “eye color.”

The user request 802 can include the genome identification 422, the phenotype data 904, the genotype data 438, or a combination thereof of the user. The phenotype data 904 and the genotype data 438 in the user request 802 can represent “eye color” and “GG” for the genome identification 422 representing “003.” The interpretation module 1222 can calculate the phenotype score 908 indicating the phenotype tendency 906 with each of the genotype data 438 for the position data 420 for the phenotype data 904 queried in the user request 802. For a specific example, the phenotype score 908 can represent “Blue Eye++” for this user.

For a different example, if there are multiple instances of the position data 420 for the genotype data 438, the interpretation module 1222 can calculate the phenotype score 908 based on aggregating the multiple instances of the phenotype score 908 for the genotype data 438, select the majority instance out of multiple instances of the phenotype score 908, or a combination thereof. For another example, based on distribution of multiple instances of the phenotype score 908 for the ethnicity, the interpretation module 1222 can calculate the phenotype score 908 for the user based on what percentile does the user belong within the distribution. Based on the phenotype score 908, the interpretation module 1222 can generate the interpretation data 902. The interpretation module 1222 can transmit the interpretation data 902 to a presentation module 1224.

The computing system 100 can include the presentation module 1224, which can be coupled to the interpretation module 1222. The presentation module 1224 displays the personal genomic data 804. For example, the presentation module 1224 can display the personal genomic data 804, the interpretation data 902, or a combination thereof.

The presentation module 1224 can display the personal genomic data 804 in a number of ways. For example, the presentation module 1224 can display the personal genomic data 804, the phenotype data 904, or a combination thereof based on the display interface 1002 of FIG. 10, the content size 1006 of FIG. 10, the user gesture 1008 of FIG. 10, or a combination thereof. For example, the display interface 1002 can include the first user interface 1118 of FIG. 11, the first display interface 1130 of FIG. 11, or a combination thereof.

For a specific example, the presentation module 1224 can display the personal genomic data 804 in two dimensional configuration on the display interface 1002. More specifically as an example, the presentation module 1224 can display the genome coordinates 1010 of FIG. 10, the phenotype data 904, the interpretation data 902, the associative research data 1014 of FIG. 10, or a combination thereof along with the personal genomic data 804. The presentation module 1224 can display the genome coordinates 1010 in the display format 1012 of FIG. 10 representing a display pin to specify the position data 420 within the personal genomic data 804 for the particular instance of the phenotype data 904, the interpretation data 902, or a combination thereof that user had requested.

For further example, the presentation module 1224 can display one or more instances of the phenotype data 904, the interpretation data 902, the associative research data 1014, or a combination thereof on the display interface 1002. More specifically as an example, the presentation module 1224 can display the phenotype data 904, the interpretation data 902, the associative research data 1014, or a combination thereof based on the display format 1012 including a display card, a list, or a combination thereof.

For another example, the presentation module 1224 can adjust the content size 1006 based on the display interface 1002. More specifically as an example, the presentation module 1224 can increase or decrease the content size 1006 represented as the font size of the personal genomic data 804 represented in alphanumeric information based on increase or decrease of the display size 1004 of FIG. 10 of the display interface 1002. For further example, the presentation module 1224 can adjust the content size 1006 based on the user gesture 1008 contacting the display interface 1002 with multiple fingers to perform the pinch action to increase or decrease the content size 1006.

For a different example, the presentation module 1224 can respond to the user gesture 1008 representing the scroll by scrolling the personal genomic data 804 displayed on the display interface 1002. More specifically as an example, the scroll can allow the user to scroll the personal genomic data 804 on the display interface 1002 up, down, left right, diagonally, or a combination thereof.

For further example, the presentation module 1224 can preload the personal genomic data 804 to minimize the delay in displaying the personal genomic data 804. More specifically as an example, the presentation module 1224 can load the personal genomic data 804 in the genomic portion 1016 of FIG. 10 to avoid loading the entire sequence of the personal genomic data 804.

The presentation module 1224 can determine the genomic portion 1016 based on the display size 1004 of the display interface 1002, the content size 1006 of the personal genomic data 804, or a combination thereof. Based on the display size 1004, the content size 1006, or a combination thereof, the presentation module 1224 can determine the genomic portion 1016 that can fit within the display interface 1002 to display the personal genomic data 804 dynamically and in real-time.

For further example, the presentation module 1224 can determine the prior instance of the genomic portion 1016, the subsequent instance of the genomic portion 1016, or a combination thereof to the genomic portion 1016 currently displayed. More specifically as an example, the presentation module 1224 can determine the prior instance of the genomic portion 1016, the subsequent instance of the genomic portion 1016, or a combination thereof to have the genomic data size 604 equivalent to the genomic data size 604 of the genomic portion 1016 currently displayed.

For a different example, the presentation module 1224 can determine the prior instance of the genomic portion 1016, the subsequent instance of the genomic portion 1016, or a combination thereof to have the genomic data size 604 smaller or larger than the genomic data size 604 of the genomic portion 1016 currently displayed. The presentation module 1224 can adjust the genomic data size 604 of the genomic portion 1016 to preload based on the content size 1006 of the personal genomic data 804, the user gesture 1008, or a combination thereof.

More specifically as an example, the user gesture 1008 can represent scrolling. The presentation module 1224 can increase or decrease the genomic data size 604 of the genomic portion 1016 to preload base on the speed of the scroll. For example, the genomic data size 604 to preload can decrease as the scroll speed increases to reduce the loading time of the genomic portion. In contrast, the genomic data size 604 to preload can increase as the scroll speed to decreases as the presentation module 1224 can have more time to load larger instance of the genomic portion 1016.

For a specific example, the presentation module 1224 can display the personal genomic data 804 in different instances of the genomic portion 1016 on the display interface 1002. The presentation module 1224 can preload the prior instance of the genomic portion 1016, the subsequent instance of the genomic portion 1016, or a combination thereof of the genomic portion 1016 currently displayed. The content size 1006 of the prior instance of the genomic portion 1016, the subsequent instance of the genomic portion 1016, or a combination thereof can be equivalent to the content size 1006 of the genomic portion 1016 currently being displayed on the display interface 1002. By preloading the prior instance of the genomic portion 1016, the subsequent instance of the genomic portion 1016, or a combination thereof, the presentation module 1224 can call the API asynchronously, minimize load time of the personal genomic data 804, allow infinite scroll, or a combination thereof.

It has been discovered that the presentation module 1224 displaying the personal genomic data 804, the phenotype data 904, or a combination thereof based on the display size 1004, the content size 1006, the user gesture 1008, or a combination thereof improves the performance of presenting the user's genomic information. By factoring the display size 1004, the computing system 100 can improve the performance to adjust the content size 1006 to be displayed of the personal genomic data 804. As a result, the computing system 100 can efficiently display the personal genomic data 804, the phenotype data 904, or a combination thereof to maximize the display interface 1002 for presenting the user's genomic information.

It has been further discovered that the presentation module 1224 preloading the personal genomic data 804 in portions improves the performance of presenting the personal genomic data 804 on the first device 102. The personal genomic data 804 can include 3 billion letters representing the genotype data 438. By preloading the genomic portion 1016, the computing system 100 can avoid loading the entire instance of the personal genomic data 804 for displaying on the first device 102. As a result, the computing system 100 can improve efficiency and performance of displaying the personal genomic data 804 on the first device 102.

The physical transformation from presenting the personal genomic data 804 including the phenotype data 904, the interpretation data 902, or a combination thereof results in the movement in the physical world, such as people using the first device 102, based on the operation of the computing system 100 by performing the user gesture 1008. As the movement in the physical world occurs, the movement itself creates additional information that is transformed from physical aspect to digital data for further presentation of the personal genomic data 804 by the computing system 100 preloading the genomic portion 1016, adjusting the content size 1006 of the personal genomic data 804 to be displayed, or a combination thereof for the continued operation of the computing system 100 and to continue the movement in the physical world.

The first software 1126 of FIG. 11 of the first device 102 of FIG. 11 can include the modules for the computing system 100. For example, the first software 1126 can include the registration module 1202, the conversion module 1204, the profile module 1206, the upload module 1208, the security module 1210, the merge module 1212, the retriever module 1220, the interpretation module 1222, and the presentation module 1224. The first control unit 1112 of FIG. 11 can execute the modules to perform the functions dynamically and in real-time.

The first control unit 1112 can execute the first software 1126 for the registration module 1202 to register the user information 204. The first control unit 1112 can execute the first software 1126 for the conversion module 1204 to generate the conversion genomic data 504. The first control unit 1112 can execute the first software 1126 for the profile module 1206 to generate the user genomic file 606. The first control unit 1112 can execute the first software 1126 for the upload module 1208 to upload the user genomic file 606. The first control unit 1112 can execute the first software 1126 for the security module 1210 to generate the encrypted genomic data 702.

The first control unit 1112 can execute the first software 1126 for the merge module 1212 to generate the format consensus file 616, the reference consensus file 620, the unification genomic file 634, or a combination thereof. The first control unit 1112 can execute the first software 1126 for the retriever module 1220 to retrieve the personal genomic data 804. The first control unit 1112 can execute the first software 1126 for the interpretation module 1222 to generate the interpretation data 902. The first control unit 1112 can execute the first software 1126 for the presentation module 1224 to display the personal genomic data 804.

The second software 1142 of FIG. 11 of the first device 102 of FIG. 11 can include the modules for the computing system 100. For example, the second software 1142 can include the registration module 1202, the conversion module 1204, the profile module 1206, the upload module 1208, the security module 1210, the merge module 1212, the retriever module 1220, the interpretation module 1222, and the presentation module 1224. The second control unit 1134 of FIG. 11 can execute the modules to perform the functions dynamically and in real-time.

The second control unit 1134 can execute the second software 1142 for the registration module 1202 to register the user information 204. The second control unit 1134 can execute the second software 1142 for the conversion module 1204 to generate the conversion genomic data 504. The second control unit 1134 can execute the second software 1142 for the profile module 1206 to generate the user genomic file 606. The second control unit 1134 can execute the second software 1142 for the upload module 1208 to upload the user genomic file 606. The second control unit 1134 can execute the second software 1142 for the security module 1210 to generate the encrypted genomic data 702.

The second control unit 1134 can execute the second software 1142 for the merge module 1212 to generate the format consensus file 616, the reference consensus file 620, the unification genomic file 634, or a combination thereof. The second control unit 1134 can execute the second software 1142 for the retriever module 1220 to retrieve the personal genomic data 804. The second control unit 1134 can execute the second software 1142 for the interpretation module 1222 to generate the interpretation data 902. The second control unit 1134 can execute the second software 1142 for the presentation module 1224 to display the personal genomic data 804.

The modules of the computing system 100 can be partitioned between the first software 1126 and the second software 1142. The second software 1142 can include the conversion module 1204, the profile module 1206, the upload module 1208, the security module 1210, the merge module 1212, the retriever module 1220, and the interpretation module 1222. The second control unit 1134 can execute modules partitioned on the second software 1142 as previously described.

The first software 1126 can include the registration module 1202 and the presentation module 1224. Based on the size of the first storage unit 1114, the first software 1126 can include additional modules of the computing system 100. The first control unit 1112 can execute the modules partitioned on the first software 1126 as previously described.

It has been discovered that the computing system 100 having different configuration of a distributed architecture to actuate each module on the first device 102 or the second device 106 enhances the capability to generate conversion genomic data 504, the user genomic file 606, the encrypted genomic data 702, the format consensus file 616, the reference consensus file 620, the unification genomic file 634, the personal genomic data 804, or a combination thereof. By having the distributed architecture including the horizontally scaled multiple instances of the second device 106 with the storage system 602 of FIG. 6 mounted, the computing system 100 can enable load distribution to process the genomic raw data 206 efficiently to reduce congestion in bottleneck in the communication path 104 and enhance the capability of the computing system 100. As a result, the computing system 100 can improve the performance to process the genomic raw data 206 for presenting the personal genomic data 804, the phenotype data 904, the interpretation data 902, or a combination thereof for efficient operation of the first device 102, the second device 106, or a combination thereof.

The first control unit 1112 can operate the first communication unit 1116 of FIG. 11 to transmit the user information 204, the conversion genomic data 504, the user genomic file 606, the encrypted genomic data 702, the format consensus file 616, the reference consensus file 620, the unification genomic file 634, the personal genomic data 804, the interpretation data 902, or a combination thereof to or from the second device 106 through the communication path 104. The first control unit 1112 can operate the first software 1126 to operate the location unit 1120. The second control unit 1134 can operate the second communication unit 1136 of FIG. 11 to transmit the user information 204, the conversion genomic data 504, the user genomic file 606, the encrypted genomic data 702, the format consensus file 616, the reference consensus file 620, the unification genomic file 634, the personal genomic data 804, the interpretation data 902, or a combination thereof to or from the first device 102 through the communication path 104.

The computing system 100 describes the module functions or order as an example. The modules can be partitioned differently. For example, the security module 1210 and the merge module 1212 can be combined. Each of the modules can operate individually and independently of the other modules. Furthermore, data generated in one module can be used by another module without being directly coupled to each other. For example, the merge module 1212 can receive the conversion genomic data 504 from the conversion module 1204. Further, one module transmitting to another module can represent one module communicating, sending, receiving, or a combination thereof the data generated to or from another module.

The modules described in this application can be hardware implementation or hardware accelerators in the first control unit 1112 or in the second control unit 1134. The modules can also be hardware implementation or hardware accelerators within the first device 102 or the second device 106 but outside of the first control unit 1112 or the second control unit 1134, respectively as depicted in FIG. 11. However, it is understood that the first control unit 1112, the second control unit 1134, or a combination thereof can collectively refer to all hardware accelerators for the modules. Furthermore, the first control unit 1112, the second control unit 1134, or a combination thereof can be implemented as software, hardware, or a combination thereof.

The modules described in this application can be implemented as instructions stored on a non-transitory computer readable medium to be executed by the first control unit 1112, the second control unit 1134, or a combination thereof. The non-transitory computer medium can include the first storage unit 1114, the second storage unit 1146 of FIG. 11, or a combination thereof. The non-transitory computer readable medium can include non-volatile memory, such as a hard disk drive, non-volatile random access memory (NVRAM), solid-state storage system (SSD), compact disk (CD), digital video disk (DVD), or universal serial bus (USB) flash memory devices. The non-transitory computer readable medium can be integrated as a part of the computing system 100 or installed as a removable portion of the computing system 100.

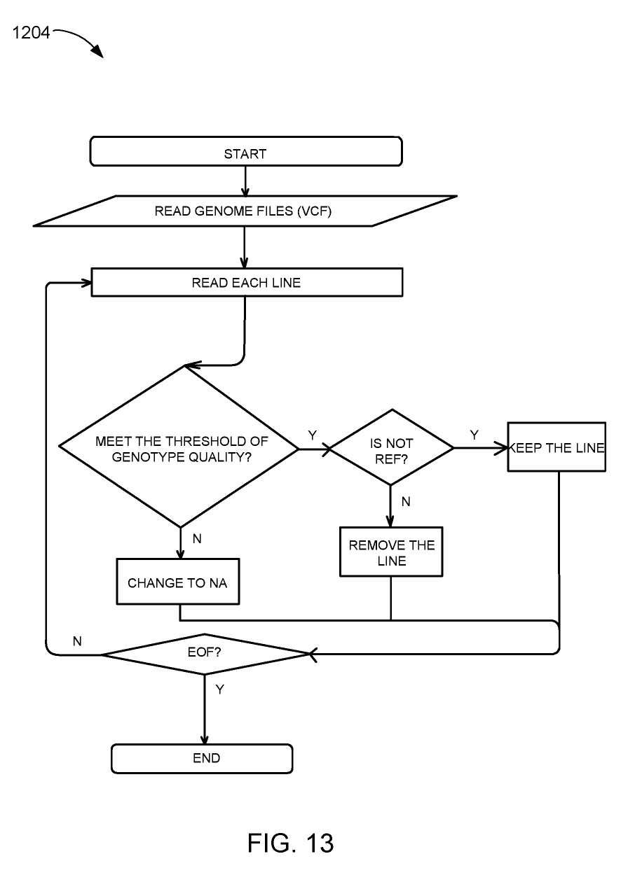

Referring now to FIG. 13, therein is shown a flow chart of the conversion module 1204. The conversion module 1204 can generate the conversion genomic data 504 of FIG. 5 in a number of ways. For example, the genomic field 406 of FIG. 4 of the genomic raw data 206 of FIG. 2 can include the chromosome data 418 of FIG. 4, the position data 420 of FIG. 4, the genotype sample 432 of FIG. 4, or a combination thereof. For further example, the genomic field 406 can include the genotype data 438 of FIG. 4 including the REF data 424 of FIG. 4, the ALT data 426 of FIG. 4, the NA data 428 of FIG. 4, or a combination thereof. The ALT data 426 can represent the comma separated list of the alternate non-reference allele(s). The conversion module 1204 can read the genomic raw data 206 line by line. More specifically as an example, the genomic raw data 206 can include multiple instances of the genomic raw line 440 of FIG. 4. The genomic raw data 206 can be in VCF format, the gVCF format, or a combination thereof.

For a specific example, the conversion module 1204 can determine whether the genotype quality 430 of FIG. 4 of the genomic raw data 206 meets or exceeds the quality threshold 512 of FIG. 5. For example, the genomic raw data 206 represented in the VCF format can include ALT data 426 and exclude the REF data 424. The genomic raw data 206 represented in the gVCF format can include the compressed instance of the REF data 424 in addition to the ALT data 426. VCF format and gVCF format normally may not include the NA data 428 to express “not available” in the genomic field 406. The conversion module 1204 can generate the conversion genomic data 504 to include the NA data 428.

More specifically as an example, if the genotype quality 430 of the genotype data 438 is less than the quality threshold 512, the conversion module 1204 can replace the genomic field 406 for the genotype data 438 with the NA data 428 or “.” (“dot”). If the genotype quality 430 of the genotype data 438 meets or exceeds the quality threshold 512, the conversion module 1204 can determine whether the genomic raw data 206 matches the reference sequence 434 based on the genotype data 438.

For a specific example, the conversion module 1204 can compare each of the genomic raw line 440 of the genomic raw data 206 to each of the genomic reference line 502 of FIG. 5 of the reference sequence 434. The conversion module 1204 can determine whether the genomic raw line 440 and the genomic reference line 502 is a match based on the genotype data 438 including a value or zero or “0/0.” In contrast, if the genotype data 438 includes a value other than zero or “0/1” for example, the conversion module 1204 can determine that the genomic raw line 440 is not a match with the genomic reference line 502. Moreover, the conversion module 1204 can determine that the genomic raw line 440 includes the genotype data 438 of the ALT data 426.

For example, if the conversion module 1204 determines that the genomic raw line 440 matches the genomic reference line 502, the conversion module 1204 can remove the genomic raw line 440. In contrast, if the conversion module 1204 determines that the genomic raw line 440 does not match with the genomic reference line 502, the conversion module 1204 can keep the genomic raw line 440. The conversion module 1204 can generate the conversion genomic data 504 including the abbreviated genomic data 506 of FIG. 5, the processed genomic data 508 of FIG. 5, or a combination thereof based on the removal of the genomic raw line 440 or not. More specifically as an example, if the genomic raw line 440 is removed, the conversion module 1204 can generate the conversion genomic data 504 as the abbreviated genomic data 506.

In contrast, if the genomic raw line 440 is not removed, the conversion module 1204 can generate the conversion genomic data 504 as the processed genomic data 508. For further example, even if the genomic raw line 440 is not removed, the genotype quality 430 can be below the quality threshold 512. As a result, the genotype data 438 may be replaced as the NA data 428. The conversion module 1204 can generate the conversion genomic data 504 as the processed genomic data 508 but including the NA data 428.

For a different example, the conversion module 1204 can generate the conversion genomic data 504 based on the genomic data size 604 of FIG. 6, the size threshold 608 of FIG. 6, or a combination thereof. For example, if the genomic data size 604 meets or exceeds the size threshold 608, the conversion module 1204 can generate the abbreviated genomic data 506 to reduce the genomic data size 604. In contrast, if the genomic data size 604 is below the size threshold 608, the conversion module 1204 can generate the processed genomic data 508.

For a different example, the conversion module 1204 can generate the conversion genomic data 504 based on the network speed 610 of FIG. 6, the speed threshold 612 of FIG. 6, or a combination thereof. For example, if the network speed 610 meets or exceeds the speed threshold 612, the conversion module 1204 can generate the abbreviated genomic data 506 to reduce the network speed 610. In contrast, if the network speed 610 is below the speed threshold 612, the conversion module 1204 can generate the processed genomic data 508.

For another example, the conversion module 1204 can generate the conversion genomic data 504 based on the sequencing result type 402 of FIG. 4. For example, if the sequencing result type 402 represents the WGS, the conversion module 1204 can generate the abbreviated genomic data 506. In contrast, if the sequencing result type 402 represents the WES, SNP, or a combination thereof, the conversion module 1204 can generate the processed genomic data 508.

It has been discovered that the conversion module 1204 generating the conversion genomic data 504 to filter the genomic raw data 206 removes the redundant instance of the genomic raw line 440. The genomic raw data 206 can have the genomic data size 604 ranging from 1 gigabyte to 10 gigabytes. And around 90% of the genomic raw data 206 can represent the REF data 424. Moreover, around 90% of the genomic raw data 206 can match the reference sequence 434, which means the genomic information representing the REF data 424 is not unique to the individual. By removing the REF data 424 from the genomic raw data 206 and keeping the ALT data 426, the NA data 428, or a combination thereof, the computing system 100 of FIG. 1 can reduce the genomic data size 604 of the genomic raw data 206 by around 90%. More specifically as an example, the computing system 100 can generate the conversion genomic data 504 representing the abbreviated genomic data 506 to exclude the REF data 424, hence maintaining the unique genomic information of the user. The computing system 100 can add back the REF data 424 to the genomic raw data 206 by referring to the chromosome data 418, the position data 420, or a combination thereof of the reference sequence 434. By removing the redundant information from the REF data 424, the computing system 100 can improve the performance and efficiency for processing and transmitting over the communication path 104 of FIG. 1 of the abbreviated genomic data 506 having the reduced instance of the genomic data size 604.

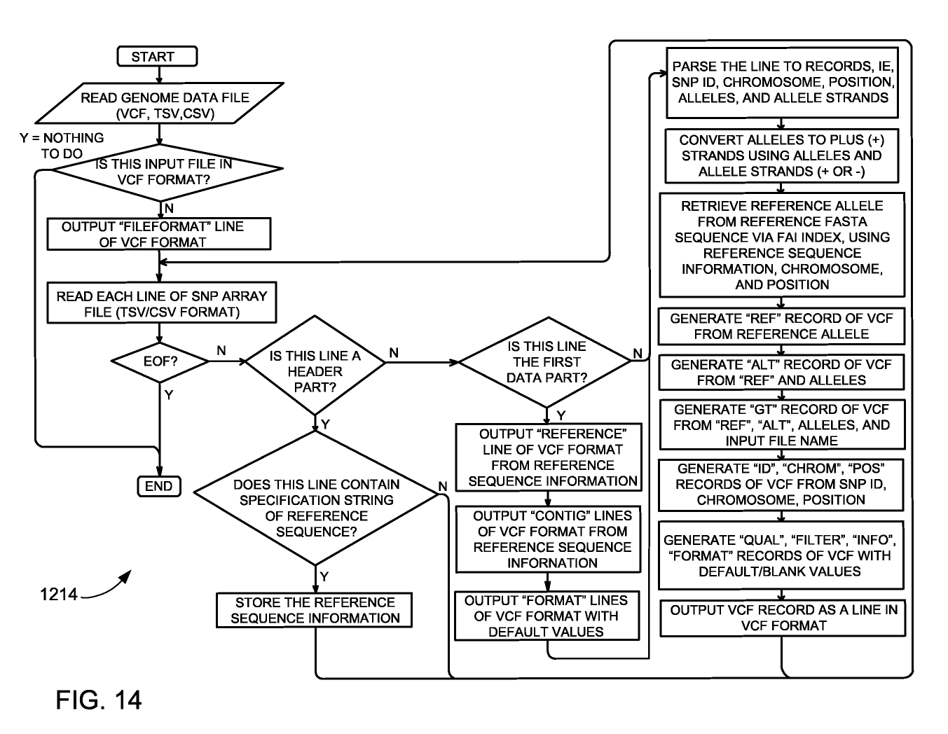

Referring now to FIG. 14, therein is shown a flow chart of the format module 1214. The format module 1214 can generate the format consensus file 616 of FIG. 6 in a number of ways. For example, the format module 1214 can determine whether the file format 404 of FIG. 4 of the conversion genomic data 504 of FIG. 5 including the abbreviated genomic data 506 of FIG. 5, the processed genomic data 508 of FIG. 5, or a combination thereof is VCF or not. Non-VCF format including SNP has no consensus format resulting in inconsistencies in the availability of the genomic field 406 of FIG. 4. The format module 1214 can generate the format consensus file 616 to unify or standardize the file format 404 to eliminate the inconsistency. If the file format 404 is determined to be VCF, the format module 1214 can generate the format consensus file 616 as is from the conversion genomic data 504.

In contrast, if the format module 1214 determines the file format 404 of the conversion genomic data 504 to represent non-VCF such as SNP array, the format module 1214 can output or designate the file format 404 of the VCF that the format consensus file 616 will be generated. For example, the format module 1214 can designate the file format 404 as “VCFv4.3.”

The conversion genomic data 504 can include the converted genomic line 510 of FIG. 5. The format module 1214 can read each line of the converted genomic line 510 of the conversion genomic data 504 until there is no more line to read from the conversion genomic data 504. If the conversion genomic data 504 is not at the end of the file, the format module 1214 can determine whether the converted genomic line 510 is the header of the block or not as discussed above. If the format module 1214 determines the converted genomic line 510 is the header, the format module 1214 can determine whether the converted genomic line 510 contains the reference sequence version 436 of FIG. 4. If the converted genomic line 510 contains the reference sequence version 436, the format module 1214 can store the reference sequence version 436 and move onto the next line of the converted genomic line 510. If the converted genomic line 510 does not contain the reference sequence version 436, the format module 1214 can move onto the next line of the converted genomic line 510 without storing.

If the format module 1214 determines the converted genomic line 510 is not the header, the format module 1214 can determine whether the converted genomic line 510 is the first data section. More specifically as an example, the format module 1214 can determine the first data section of the converted genomic line 510 based on the first line of data after the genomic field 406.

Continuing with the example, if the converted genomic line 510 is the first data section, the format module 1214 can generate the reference data 408 of FIG. 4 representing “reference” for example in the file format 404 of VCF based on the reference sequence version 436 from the converted genomic line 510. The format module 1214 can generate the contig data 410 of FIG. 4 representing “contig” for example in the file format 404 of VCF based on the reference sequence version 436 from the converted genomic line 510. The format module 1214 can generate the field format 412 of FIG. 4 representing “FORMAT” for example in the file format 404 of VCF with default values.

Subsequent to generating the field format 412 representing “FORMAT” or the converted genomic line 510 not being the first data section, the format module 1214 can parse the converted genomic line 510. More specifically as an example, the format module 1214 can parse the genomic field 406 of the converted genomic line 510. For a specific example, the format module 1214 can parse the genomic field 406 including the genome identification 422 of FIG. 4, chromosome data 418 of FIG. 4, the position data 420 of FIG. 4, the genotype data 438 of FIG. 4, or a combination thereof. The genome identification 422 can represent the SNP identification. The converted genomic line 510 can include multiple fields for the genomic field 406 including the genotype data 438. More specifically as an example, the genotype data 438 can represent the allele and/or allele strands.

The format module 1214 can convert the genotype data 438 representing the allele strand between positive (“+”) or negative (“−”) within the converted genomic line 510. More specifically as an example, when the allele strand of the conversion genomic data 504 has the same strand as the allele strand for the reference sequence 434 of FIG. 4, the genotype data 438 can represent “+.” In contrast, for the conversion genomic data 504 representing SNP array having the reverse strand as the allele strand for the reference sequence 434, the genotype data 438 can represent “−.” For example, the allele strand for the reference sequence 434 can represent “AGC” and the reverse strand would be “TCG” according to the DNA pairing for double strand. The format module 1214 can convert the genotype data 438 that is “−” into “+” for the file format 404 representing VCF. More specifically as an example, the format module 1214 can convert the genotype data 438 of reverse strand of “TCG” into “AGC.”

The format module 1214 can retrieve the genotype data 438 representing the reference allele from the reference sequence 434 in the file format 404 of FASTA using the fai index. More specifically as an example, the format module 1214 can retrieve the reference allele based on the reference sequence version 436, the chromosome data 418, the position data 420, or a combination thereof.

Continuing with the example, the format module 1214 can generate the genomic field 406 represented as “REF” for the REF data 424 of FIG. 4 in the file format 404 of VCF from the reference allele retrieved. Further, the format module 1214 can generate the genomic field 406 represented as “ALT” for the ALT data 426 of FIG. 4 in the file format 404 of VCF from the REF data 424, the genotype data 438 of the converted genomic line 510, or a combination thereof. More specifically as an example, the genotype data 438 can represent allele from the converted genomic line 510. For further example, the format module 1214 can compare the genotype data 438 of the converted genomic line 510 with the REF data 424 of the reference sequence 434. If the genotype data 438 is different from the REF data 424, the format module 1214 can determine the genotype data 438 as the ALT data 426.

For a different example, the format module 1214 can generate the genotype sample 432 of FIG. 4 based on the REF data 424, the ALT data 426, or a combination thereof. For example, the REF data 424 can represent “A” and the ALT data 426 can represent “T.” Since the REF data 424 and the ALT data 426 are different, the format module 1214 can generate the genotype sample 432 as “0/1.” The format module can populate the genotype sample 432 in the genomic field 406 for the genotype sample 432 following the genomic field 406 represented as “FORMAT.” Continuing with the example, the format module 1214 can generate the genomic field 406 for the genotype sample 432 in the file format 404 of VCF from the REF data 424, the ALT data 426, the genotype data 438 of the converted genomic line 510 representing the allele, the filename of the conversion genomic data 504, or a combination thereof.

Further, the format module 1214 can generate the genomic field 406 represented as “ID” for the genome identification 422, the genomic field 406 represented as “CHROM” for the chromosome data 418, the genomic field 406 represented as “POS” for the position data 420, or a combination thereof in the file format 404 of VCF from the genome identification 422, chromosome data 418, the position data 420, or a combination thereof of the converted genomic line 510.

Continuing with the example, the format module 1214 can generate the genomic field 406 for the genotype quality 430 of FIG. 4 as “QUAL,” the filter status 414 of FIG. 4 as “FILTER,”, the additional information 416 of FIG. 4 as “INFO,” the field format 412 as “FORMAT,” or a combination thereof based on the file format 404 specified for VCF. The format module 1214 can generate the genomic field 406 for “QUAL,” “FILTER,” “INFO,” “FORMAT,” or a combination thereof with default, blank, or a combination thereof values.

As a result, the format module 1214 can generate the VCF formatted line 618 of FIG. 6 including multiple fields as represented above for the genomic field 406 based on converting the converted genomic line 510 according to the file format 404 representing VCF. The format module 1214 can repeat the above process until the end of file where the converted genomic line 510 is no longer available for reformatting into VCF. The format module 1214 can aggregate the multiple instances of the VCF formatted line 618 to generate the format consensus file 616.

It has been discovered that the format module 1214 generating the format consensus file 616 improves the efficiency of the computing system 100 of FIG. 1 analyzing the genomic raw data 206 of FIG. 2. More specifically as an example, by generating the format consensus file 616, the computing system 100 can standardize the genomic raw data 206 into specified instance of the file format 404. By having the file format 404 standardized, the computing system 100 can eliminate inconsistencies arising from missing instance of the genomic field 406 when two different instances of the file format 404 are compared. As a result, the computing system 100 can improve the performance to analyze the genomic raw data 206 as irregularities from different instances of the file format 404 are eliminated.

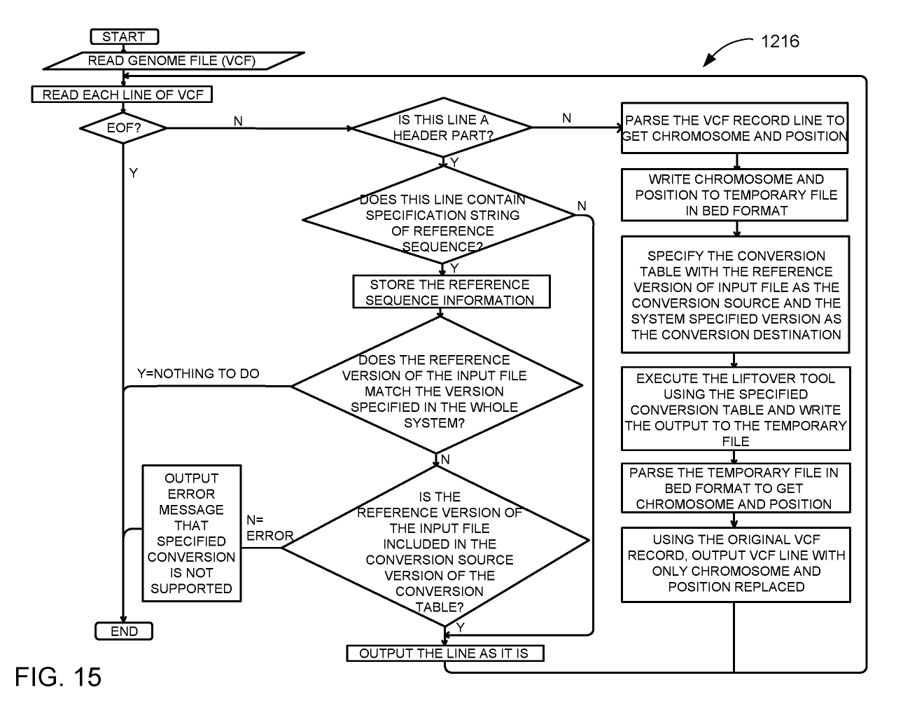

Referring now to FIG. 15, therein is shown a flow chart of the reference module 1216. The reference module 1216 can generate the reference consensus file 620 of FIG. 6 in a number of ways. For example, the reference module 1216 can read in the conversion genomic data 504 of FIG. 5, the format consensus file 616 of FIG. 6, or a combination thereof. Further, the reference module 1216 can read in the converted genomic line 510 of FIG. 5, the VCF formatted line 618 of FIG. 6, or a combination thereof. If the converted genomic line 510, the VCF formatted line 618, or a combination thereof are not at end of file, the reference module 1216 can determine whether the read in portion of the converted genomic line 510, the VCF formatted line 618, or a combination thereof represents header or not.

If the reference module 1216 determined the converted genomic line 510, the VCF formatted line 618, or a combination thereof as the header, the reference module 1216 can determine whether the converted genomic line 510, the VCF formatted line 618, or a combination thereof includes the reference sequence version 436 of FIG. 4. If the reference module 1216 determined that the converted genomic line 510, the VCF formatted line 618, or a combination thereof does not include the reference sequence version 436, then the reference module 1216 can read the subsequent line of the converted genomic line 510, the VCF formatted line 618, or a combination thereof. If the reference module 1216 determined that the reference sequence version 436 is in the header, the reference module 1216 can store the reference sequence version 436 of the converted genomic line 510, the VCF formatted line 618, or a combination thereof in the first storage unit 1114 of FIG. 11, the second storage unit 1146 of FIG. 11, or a combination thereof.

Continuing with the example, the reference module 1216 can determine whether the reference sequence version 436 of the converted genomic line 510, the VCF formatted line 618, or a combination thereof matches with the system reference version 622 of FIG. 6 or not. If the reference sequence version 436 and the system reference version 622 matches, the reference module 1216 can generate or include the converted genomic line 510, the VCF formatted line 618, or a combination thereof as part of the reference consensus file 620.

In contrast, if the reference sequence version 436 and the system reference version 622 does not match, the reference module 1216 can determine whether the reference sequence version 436 is included as the conversion source version 626 of FIG. 6 stored in the conversion table 624 of FIG. 6. If the reference sequence version 436 is included as the conversion source version 626, the reference module 1216 can generate or include the converted genomic line 510, the VCF formatted line 618, or a combination thereof as part of the reference consensus file 620. If the reference sequence version 436 is not included as the conversion source version 626, the reference module 1216 can generate the message 628 of FIG. 6 indicating an error that the reference sequence version 436 is not supported.

If the read in portion of converted genomic line 510, the VCF formatted line 618, or a combination thereof represents is not the header, the reference module 1216 can parse the converted genomic line 510, the VCF formatted line 618, or a combination thereof to obtain the chromosome data 418 of FIG. 4, the position data 420 of FIG. 4, or a combination thereof. The reference module 1216 can write the chromosome data 418, the position data 420, or a combination thereof to the temporary file 632 of FIG. 6 in the file format 404 of FIG. 4 of Browser Extensible Data (BED) format as an example. The reference module 1216 can specify the conversion table 624 with the reference sequence version 436 as a conversion source and the system reference version 622 as a conversion destination. The conversion table 624 can include the version difference 630 of FIG. 6 between the reference sequence version 436 and the system reference version 622.

The reference module 1216 can generate the reference consensus file 620 based on the version difference 630. More specifically as an example, based on the version difference 630, the reference module 1216 can convert the converted genomic line 510, the VCF formatted line 618, or a combination thereof by reformatting the file format 404 for the reference sequence version 436 into the file format 404 for the system reference version 622 and output to the temporary file 632. The reference module 1216 can parse the temporary file 632 in the BED format to obtain the chromosome data 418, the position data 420, or a combination thereof. The reference module 1216 can generate the reference consensus file 620 including the chromosome data 418, the position data 420 replaced according to the system reference version 622 based on the converted genomic line 510, the VCF formatted line 618, or a combination thereof.

It has been discovered that the reference module 1216 generating the reference consensus file 620 improves the efficiency of the computing system 100 of FIG. 1 analyzing the genomic raw data 206 of FIG. 2. More specifically as an example, by generating the reference consensus file 620, the computing system 100 can standardize the genomic raw data 206 into specified version of the reference sequence version 436. By having the reference sequence version 436 standardized, the computing system 100 can eliminate inconsistencies arising from different configurations of the genomic field 406 when the reference sequence 434 is different. As a result, the computing system 100 can improve the performance to analyze the genomic raw data 206 as irregularities from different instances of the reference sequence version 436 are eliminated.

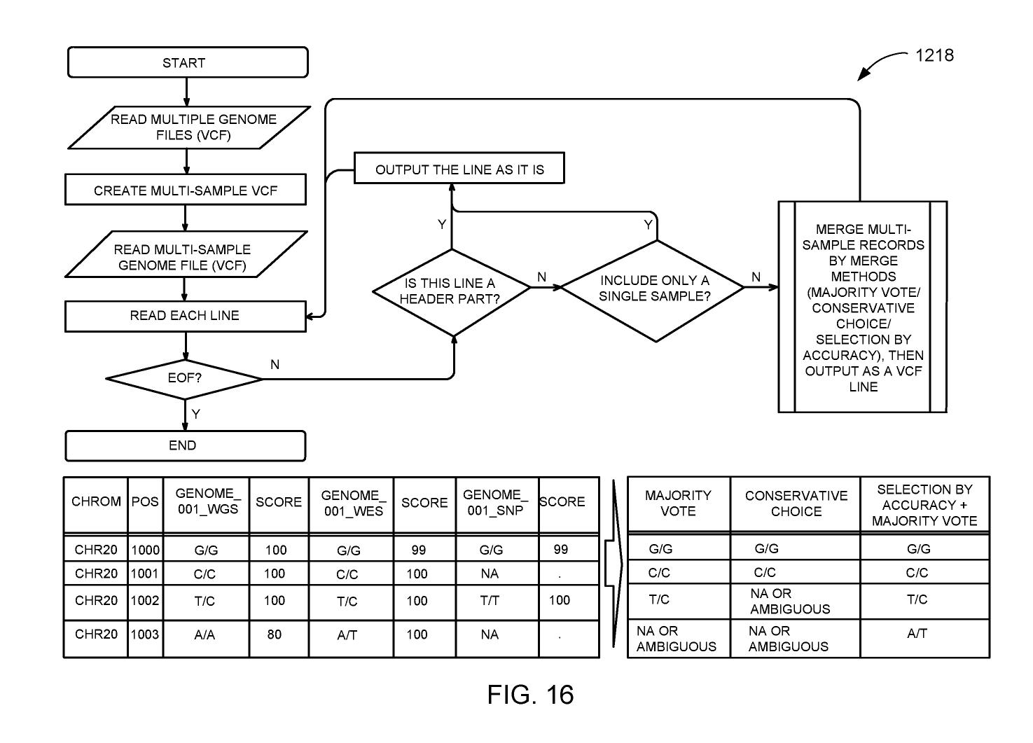

Referring now to FIG. 16, therein is shown a flow chart of the multi module 1218. The multi module 1218 can generate the unification genomic file 634 of FIG. 6 in a number of ways. For example, the multi module 1218 can read in multiple files represented as the conversion genomic data 504 of FIG. 5, the format consensus file 616 of FIG. 6, the reference consensus file 620 of FIG. 6, or a combination thereof.

The multi module 1218 can generate the multi-sample file 638 of FIG. 6 including different instances of the genotype sample 432 of FIG. 4 based on aggregating the conversion genomic data 504 of FIG. 5, the format consensus file 616 of FIG. 6, the reference consensus file 620 of FIG. 6, or a combination thereof. More specifically as an example, the multi module 1218 can generate the multi-sample file 638 based on creating a set of union by combining multiple different instances of the conversion genomic data 504, the format consensus file 616, the reference consensus file 620, or a combination thereof. The multi module 1218 can generate the set of union by combining the various instances of the conversion genomic data 504, the format consensus file 616, the reference consensus file 620, or a combination thereof sharing the genomic field 406 of FIG. 4 of the chromosome data 418 of FIG. 4, the position data 420 of FIG. 4, or a combination thereof.

The multi module 1218 can generate the multi-sample file 638 including the chromosome data 418, the position data 420, the genotype sample 432, or a combination thereof. More specifically as an example, the multi-sample file 638 can include various instances of the genotype sample 432 of the same user. The various instances of the genotype sample 432 can be derived from different instances of user genomic file 606 of FIG. 6 represented in various instances of the file format 404 of FIG. 4 including WGS, WES, SNP array, or a combination thereof. As discussed above, the multi module 1218 can generate the multi-sample file 638 representing a set of union of various instances of the genotype sample 432 sharing the same instance of the chromosome data 418, the position data 420, or a combination thereof.

The multi module 1218 can read the multi-sample file 638 including the multi-sample line 640 of FIG. 6. The multi module 1218 can read each line of the multi-sample line 640. Unless the multi module 1218 reaches the end of file, the multi module 1218 can determine whether the multi-sample line 640 read in represents the header or not. If the multi-sample line 640 represents the header, the multi module 1218 can read the next line of the multi-sample line 640.

If the multi-sample line 640 is not the header, the multi module 1218 can determine whether there is one instance of the genotype sample 432 or not within the multi-sample line 640. If there is only one instance of the genotype sample 432, the multi module 1218 can output the multi-sample line 640 as is the unification genomic file 634. In contrast, if there are multiple samples of the genotype sample 432 within the multi-sample line 640, the multi module 1218 can merge the multiple samples into one sample of the genotype sample 432 to generate the unification genomic file 634.

The multi module 1218 can generate the unification genomic file 634 in a number of ways. For example, multiple different instances of the user genomic file 606 can be uploaded for the user of computing system 100. More specifically as an example, one of the user genomic file 606 can include the SNP array for the user in one upload. Another instance of the user genomic file 606 including the WGS can be uploaded for the same user. And a different instance of the user genomic file 606 including the WES can be also uploaded for the same user. The different instances of the user genomic file 606 can be generated by different instance of the gene mapping device, the time period 652 of FIG. 6, or a combination thereof. As a result, different instances of the genotype sample 432 can be generated for the same user. As discussed above, the user genomic file 606 can be converted as the conversion genomic data 504, the format consensus file 616, the reference consensus file 620, or a combination thereof.

Continuing with the example, the multi module 1218 can generate the unification genomic file 634 based on merging different instances of the multi-sample file 638 for the same user in a number of ways. More specifically as an example, the multi module 1218 can generate the unification genomic file 634 based on the merge policy 642 of FIG. 6 including the majority vote policy 644 of FIG. 6, the conservative choice policy 646 of FIG. 6, the accuracy policy 648 of FIG. 6, the time period policy 650 of FIG. 6, or a combination thereof.

The merge policy 642 can be configured within the computing system 100. More specifically as an example, the determination of which instance of the merge policy 642 to be applied can be updated dynamically and in real-time. Details regarding the application of the merge policy 642 are discussed below.

For a specific example, the multi module 1218 can generate the unification genomic file 634 based on the majority vote policy 644. More specifically as an example, the multi-sample file 638 can include multiple samples of the genotype sample 432 such as WGS, WES, SNP, or a combination thereof. The genotype sample 432 for WGS can represent “T/C.” The genotype sample 432 for WES can represent “T/C.” The genotype sample 432 for SNP can represent the “T/T.” Based on the majority vote policy 644, since the 2 out of 3 samples of the genotype sample 432 are “T/C,” the multi module 1218 can generate the unification genomic file 634 including the genotype sample 432 unified as “T/C.” If a majority number of samples cannot be determined, the multi module 1218 can generate the unification genomic file 634 including the genotype sample 432 as the NA data 428 based on the majority vote policy 644. For a different example, if a majority number of samples cannot be determined, the multi module 1218 can generate the unification genomic file 634 based on the accuracy policy 648.

For a different example, the multi module 1218 can generate the unification genomic file 634 based on the conservative choice policy 646. Continuing with the previous example, the genotype sample 432 for WGS can represent “T/C.” The genotype sample 432 for WES can represent “T/C.” The genotype sample 432 for SNP can represent the “T/T.” Based on the conservative choice policy 646, if there are at least 2 samples of the genotype sample 432 having different results, the multi module 1218 can generate the unification genomic file 634 including the genotype sample 432 as the NA data 428 or ambiguous.

For a different example, the multi module 1218 can generate the unification genomic file 634 based on the accuracy policy 648. The genotype sample 432 for WGS can represent “A/A” having the genotype quality 430 of “80.” The genotype sample 432 for WES can represent “A/T” having the genotype quality 430 of “100.” The genotype sample 432 for SNP can represent the NA data 428 thus without the genotype quality 430. The genotype quality 430 can represent the value from the genomic field 406 representing “QUAL” of VCF, the “DP” value within the genotype sample 432 representing combined depth across samples, or a combination thereof. Based on the accuracy policy 648, if there are at least 2 samples of the genotype sample 432, the multi module 1218 can generate the unification genomic file 634 including the genotype sample 432 with having the highest instance of the genotype quality 430 representing “A/T.”

For a different example, the multi module can generate the unification genomic file 634 based on the time period policy 650. Continuing with the previous example, the genotype sample 432 for WGS can represent “T/C.” The genotype sample 432 for WES can represent “T/C.” The genotype sample 432 for SNP can represent the “T/T.” Based on the time period policy 650, the multi module 1218 can generate the unification genomic file 634 including the genotype sample 432 having the most current instance of the time period 652, the oldest instance of the time period 652, the time period 652 that is closest to the average instance of multiple different instances of the time period 652, or a combination thereof.

The multi module 1218 can generate the multi-sample file 638 by aggregating all instances of the conversion genomic data 504, the format consensus file 616, the reference consensus file 620, or a combination thereof available prior to reading each of the multi-sample line 640. For a different example, the multi module 1218 can generate the multi-sample file 638 by reading in each of the conversion genomic data 504, the format consensus file 616, the reference consensus file 620, or a combination thereof sequentially based on the chromosome data 418, the position data 420, or a combination thereof. As one example, the multi module 1218 can generate the unification genomic file 634 only if the conversion genomic data 504, the format consensus file 616, the reference consensus file 620, or a combination thereof that are read in share the same instance of the chromosome data 418, the position data 420, or a combination thereof.

It has been discovered that the multi module 1218 generating the unification genomic file 634 based on the merge policy 642 improves the performance and efficiency of presenting the user's genomic information. Each instance of the user's genomic information can have the genomic content size 1006 ranging from a gigabyte to multi-gigabytes. Having multiple different instances of the user's genomic information, the computing system 100 of FIG. 1 can require significant amount of resources to process each instance of the genomic information. By unifying various instances of the user's genomic information into one instance of the unification genomic file 634, the computing system 100 can reduce resource required to process the genomic information. As a result, the computing system 100 can allocate the additional computer resource to other functionalities to improve the performance of the computing system 100.

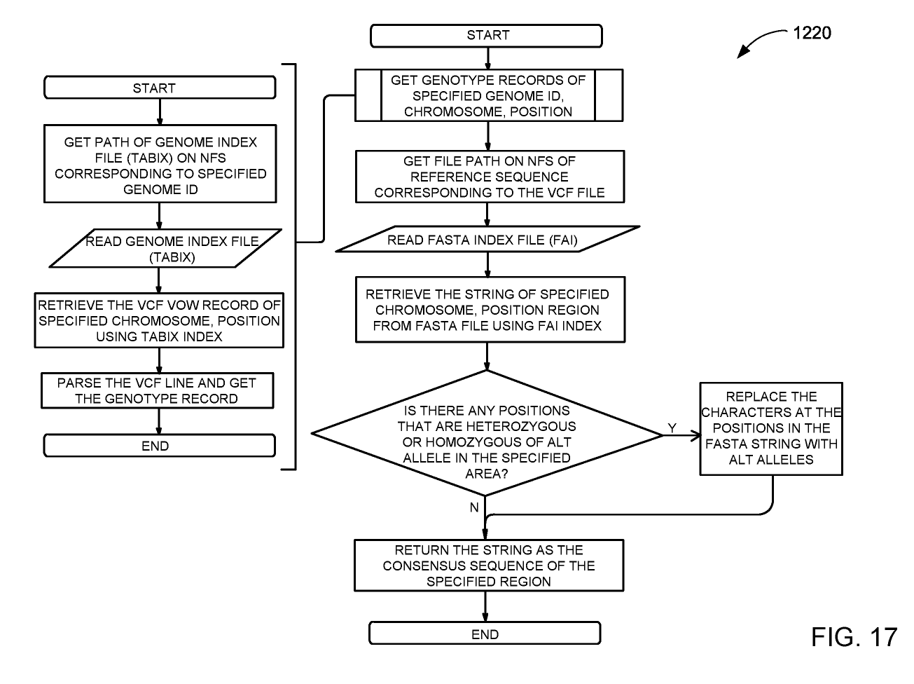

Referring now to FIG. 17, therein is shown a first flow chart of the retriever module 1220. The retriever module 1220 can retrieve the personal genomic data 804 of FIG. 8 in a number of ways. For example, the retriever module 1220 can retrieve the personal genomic data 804 including the genotype data 438 of FIG. 4 based on decrypting the encrypted instance of the unification genomic file 634 of FIG. 6 including the unified genomic line 636 of FIG. 6, the encrypted index 706 of FIG. 7, or a combination thereof. The retriever module 1220 can decrypt the unification genomic file 634 similarly as the security module 1210 decrypting the encrypted genomic data 702 of FIG. 7. The retriever module 1220 can generate the decrypted index 714 of FIG. 7 similarly as the security module 1210 can generate the decrypted index 714.

For further example, the retriever module 1220 can retrieve the genotype data 438 based on obtaining the file path of the decrypted index 714 representing the tabix index on the storage system 602 of FIG. 6 representing the NFS. The user request 802 of FIG. 8 can include the genome identification 422 of FIG. 4, the chromosome data 418 of FIG. 4, the position data 420 of FIG. 4, or a combination thereof for the genotype data 438 that the user is requesting. For a specific example, the user request 802 can include the genome identification 422 of “0,” the chromosome data 418 of “chr1,” and the position data 420 ranging from the start position 806 of FIG. 8 of “72,017” to the end position 808 of FIG. 8 of “72,117” under the 0-based index.

More specifically as an example, the decrypted index 714 representing the tabix index can correspond to the specified instance of the genome identification 422 that the user is requesting. The retriever module 1220 can retrieve the unified genomic line 636 corresponding to the chromosome data 418, the position data 420, or a combination thereof based on the tabix index that corresponds to the specified instance of the genome identification 422. The presentation module 1224 can retrieve the genotype data 438 based on parsing the unified genomic line 636.

For a different example, the retriever module 1220 can retrieve the consensus sequence 810 of FIG. 8 based on the genotype data 438, the genome identification 422, the chromosome data 418, the position data 420, or a combination thereof. The process to retrieve the consensus sequence 810 can include the process to retrieve the genotype data 438 as discussed above. More specifically as an example, the retriever module 1220 can retrieve the genotype data 438 based on the genome identification 422, the chromosome data 418, the position data 420, or a combination thereof. Further, the retriever module 1220 can obtain the file path on the NFS of the reference sequence 434 of FIG. 4 corresponding to the unification genomic file 634. Based on reading the FASTA fai index, the retriever module 1220 can retrieve the sequence string 812 of FIG. 8 specified in the chromosome data 418, the position data 420 ranging from the start position 806 to the end position 808, or a combination thereof of the reference sequence 434. The sequence string 812 can be in FASTA format.

The retriever module 1220 can determine whether the unified genomic line 636 includes the ALT data 426 of FIG. 4 representing the ALT allele within the chromosome data 418, the position data 420, or a combination thereof when compared to the sequence string 812 of the reference sequence 434. For example, the retriever module 1220 can determine whether unified genomic line 636 includes the ALT data 426 for heterozygous or homozygous. More specifically as an example, when the unified genomic line 636 is for heterozygous, the retriever module 1220 can determine whether the unified genomic line 636 includes one of the allele as ALT data 426 or not. In contrast, when the unified genomic line 636 is for homozygous, the retriever module 1220 can determine whether the unified genomic line 636 includes two alleles that are ALT data 426 or not.

If the unified genomic line 636 does not include the ALT data 426, the retriever module 1220 can return the sequence string 812 as the consensus sequence 810 for the chromosome data 418, the position data 420, or a combination thereof. For further example, if the unified genomic line 636 does not include the ALT data 426 but includes that NA data 428 of FIG. 4, the retriever module 1220 can return the sequence string 812 as the consensus sequence 810 for the chromosome data 418, the position data 420, or a combination thereof to see the REF data 424 within the sequence string 812.

If the unified genomic line 636 includes the ALT data 426, the retriever module 1220 can replace the genotype data 438 within the sequence string 812 for the position data 420 that is different between the unified genomic line 636 versus the sequence string 812 with ALT data 426. More specifically as an example, the retriever module 1220 can replace the character at the position in the sequence string 812 with the ALT allele(s). As a result, the retriever module 1220 can return the sequence string 812 with the ALT data 426 replaced as the consensus sequence 810. Subsequently, the retriever module 1220 can generate the personal genomic data 804 based on the sequence string 812.

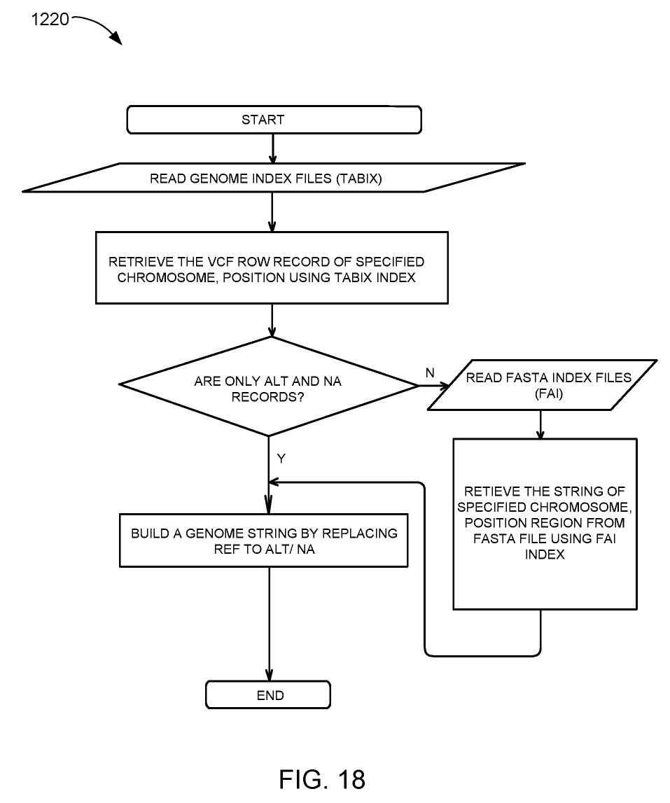

Referring now to FIG. 18, therein is shown a second flow chart of the retriever module 1220. For example, the retriever module 1220 can retrieve the personal genomic data 804 of FIG. 8 based on the unification genomic file 634 of FIG. 6 including the unified genomic line 636 of FIG. 6 generated from the abbreviated genomic data 506 of FIG. 5. Based on the decrypted index 714 of FIG. 7 representing the tabix index, the retriever module 1220 can retrieve the unified genomic line 636 within the specified instance of the chromosome data 418 of FIG. 4, the position data 420 of FIG. 4 ranging from the start position 806 of FIG. 8 to the end position 808 of FIG. 8, or a combination thereof as specified in the user request 802. More specifically as an example, the retriever module 1220 can determine whether the unified genomic line 636 is retrievable based on each of the position data 420 specified.

If the retriever module 1220 can retrieve the unified genomic line 636 for each of the position data 420, the retriever module 1220 can generate personal genomic data 804 by concatenating the ALT data 426 of FIG. 4, the NA data 428 of FIG. 4, or a combination thereof. If the retriever module 1220 cannot retrieve the unified genomic line 636 for each of the position data 420, based on reading the FASTA fai index, the retriever module 1220 can retrieve the sequence string 812 of FIG. 8 specified in the chromosome data 418, the position data 420 ranging from the start position 806 to the end position 808, or a combination thereof of the reference sequence 434. The sequence string 812 can be in FASTA format. The retriever module 1220 can generate the personal genomic data 804 by replacing the REF data 424 of FIG. 4 in the sequence string 812 with the ALT data 426, the NA data 428, or a combination thereof for each of the position data 420 including the REF data 424.

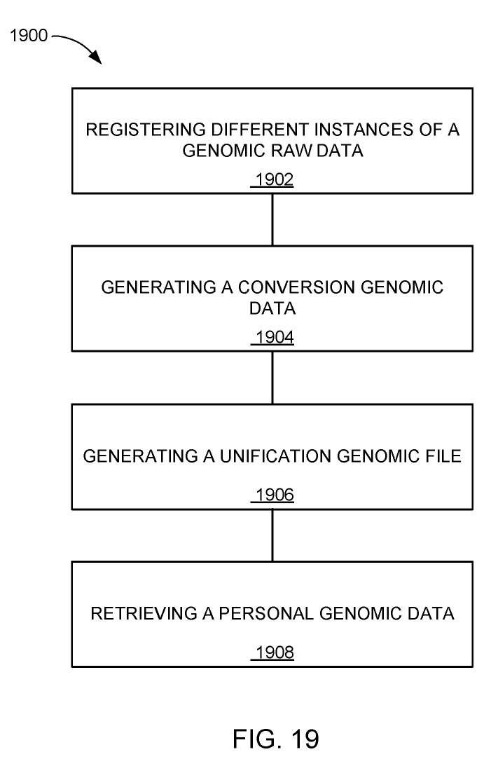

Referring now to FIG. 19, therein is shown a flow chart of a method 1900 of operation of the computing system 100 in a further embodiment of the present invention. The method 1900 includes: registering different instances of a genomic raw data for a user profile in a block 1902; generating a conversion genomic data for each of the genomic raw data by removing a genomic raw line of the genomic raw data for reducing a genomic data size in a block 1904; generating a unification genomic file with a control unit based on a merge policy for merging different instances of a genotype sample from each of the conversion genomic data in a block 1906; and retrieving a personal genomic data based on the unification genomic file for presenting an interpretation data on a device in a block 1908.

The resulting method, process, apparatus, device, product, and/or system is straightforward, cost-effective, uncomplicated, highly versatile, accurate, sensitive, and effective, and can be implemented by adapting known components for ready, efficient, and economical manufacturing, application, and utilization. Another important aspect of the present invention is that it valuably supports and services the historical trend of reducing costs, simplifying systems, and increasing performance. These and other valuable aspects of the present invention consequently further the state of the technology to at least the next level.

While the invention has been described in conjunction with a specific best mode, it is to be understood that many alternatives, modifications, and variations will be apparent to those skilled in the art in light of the aforegoing description. Accordingly, it is intended to embrace all such alternatives, modifications, and variations that fall within the scope of the included claims. All matters hithertofore set forth herein or shown in the accompanying drawings are to be interpreted in an illustrative and non-limiting sense.

What is claimed is:

1. A method of operation of a computing system comprising:
registering different instances of a genomic raw data for a user profile;
generating a conversion genomic data for each of the genomic raw data by removing a genomic raw line of the genomic raw data for reducing a genomic data size;
generating a unification genomic file with a control unit based on a merge policy for merging different instances of a genotype sample from each of the conversion genomic data; and
retrieving a personal genomic data based on the unification genomic file for presenting a phenotype data, an interpretation data, or a combination thereof on a device.

2. The method as claimed in claim 1 further comprising generating a format consensus file to unify a file format of each of the conversion genomic data.

3. The method as claimed in claim 1 further comprising generating a reference consensus file to unify a reference sequence version of each of the conversion genomic data.

4. The method as claimed in claim 1 further comprising preloading a genomic portion based on a display size for controlling the personal genomic data to be displayed on the device.

5. The method as claimed in claim 1 further comprising mounting a storage system for horizontally scaling multiple instances of a server to process the genomic raw data.

6. The method as claimed in claim 1 wherein generating the unification genomic file includes generating the unification genomic file based on a majority vote policy for selecting the genotype sample constituting a majority number.

7. The method as claimed in claim 1 wherein generating the unification genomic file includes generating the unification genomic file based on a conservative choice policy for assigning a not available (NA) data when different instances of the genotype sample are available.

8. The method as claimed in claim 1 wherein generating the unification genomic file includes generating the unification genomic file based on an accuracy policy for selecting the genotype sample according to a genotype quality.

9. The method as claimed in claim 1 wherein generating the unification genomic file includes generating the unification genomic file based on a time period policy for selecting the genotype sample according to when the genotype sample was sequenced.

10. The method as claimed in claim 1 wherein generating the unification genomic file includes generating the unification genomic file based on creating a multi-sample file according to a set of union for sharing a chromosome data, a position data, or a combination thereof.

11. A computing system comprising:
a control unit for:
registering different instances of a genomic raw data for a user profile;
generating a conversion genomic data for each of the genomic raw data by removing a genomic raw line of the genomic raw data for reducing a genomic data size;
generating a unification genomic file based on a merge policy for merging different instances of a genotype sample from each of the conversion genomic data;
retrieving a personal genomic data based on the unification genomic file; and
a communication unit, coupled to the control unit, for transmitting the personal genomic data for presenting a phenotype data, an interpretation data, or a combination thereof on a device.

12. The system as claimed in claim 11 wherein the control unit is for generating a format consensus file to unify a file format of each of the conversion genomic data.

13. The system as claimed in claim 11 wherein the control unit is for generating a reference consensus file to unify a reference sequence version of each of the conversion genomic data.

14. The system as claimed in claim 11 wherein the control unit is for preloading a genomic portion based on a display size for controlling the personal genomic data to be displayed on the device.

15. The system as claimed in claim 11 wherein the control unit is for mounting a storage system for horizontally scaling multiple instances of a server to process the genomic raw data.

16. A non-transitory computer readable medium including instructions for execution, the instructions comprising:
registering different instances of a genomic raw data for a user profile;
generating a conversion genomic data for each of the genomic raw data by removing a genomic raw line of the genomic raw data for reducing a genomic data size;
generating a unification genomic file with a control unit based on a merge policy for merging different instances of a genotype sample from each of the conversion genomic data; and
retrieving a personal genomic data based on the unification genomic file for presenting a phenotype data, an interpretation data, or a combination thereof on a device.

17. The non-transitory computer readable medium as claimed in claim 16 further comprising generating a format consensus file to unify a file format of each of the conversion genomic data.

18. The non-transitory computer readable medium as claimed in claim 16 further comprising generating a reference consensus file to unify a reference sequence version of each of the conversion genomic data.

19. The non-transitory computer readable medium as claimed in claim 16 further comprising preloading a genomic portion based on a display size for controlling the personal genomic data to be displayed on the device.

20. The non-transitory computer readable medium as claimed in claim 16 further comprising mounting a storage system for horizontally scaling multiple instances of a server to process the genomic raw data.
